(19) United States
(12) Patent Application Publication
(10) Pub. No.: US 2024/0287529 A1
(43) Pub. Date: Aug. 29, 2024

(54) CPG OLIGODEOXYNUCLEOTIDES AS IMMUNOSTIMULANTS OF FISH

(71) Applicants: THE STATE OF ISRAEL, MINISTRY OF AGRICULTURE & RURAL DEVELOPMENT, AGRICULTURAL RESEARCH ORGANIZATION, Rishon Lezion (IL); NIBN, The National Institute for Biotechnology in the Negev Ltd., Beer Sheva (IL); ISRAEL OCEANOGRAPHIC AND LIMNOLOGICAL RESEARCH, LTD., Eilat (IL)

(72) Inventors: Galit SHARON, Be'er Ora (IL); Ari Angel PORGADOR, Lehavim (IL); Shay RAVID -PERETZ, Eilat (IL)

(73) Assignees: THE STATE OF ISRAEL, MINISTRY OF AGRICULTURE & RURAL DEVELOPMENT, AGRICULTURAL RESEARCH ORGANIZATION, Rishon Lezion (IL); NIBN, The National Institute for Biotechnology in the Negev Ltd., Beer Sheva (IL); ISRAEL OCEANOGRAPHIC AND LIMNOLOGICAL RESEARCH, LTD., Eilat (IL)

(21) Appl. No.: 18/571,745

(22) PCT Filed: Jun. 19, 2022
(86) PCT No.: PCT/IL2022/050655

§ 371 Date: Dec. 19, 2023

Related U.S. Application Data
(60) Provisional application No. 63/212,676, filed on Jun. 20, 2021.

(51) Int. Cl. C12N 15/117; A61K 31/7125; A61P 37/04

(52) U.S. Cl. CPC C12N 15/117; A61K 31/7125; A61P 37/04

(57) ABSTRACT

The present disclosure provides CpG oligodeoxynucleotides (ODNs) as immunogenic molecules, and their use in methods of initiating or promoting innate or adaptive immunity in fish or increasing an immune response against an antigen in fish.

### SEQUENCE LISTING STATEMENT

The instant application contains a Sequence Listing which has been submitted electronically in ASCII format and is hereby incorporated by reference in its entirety. Said ASCII copy, created on Jun. 19, 2022, is named P-606497-PC-SL.txt and is 3,054 bytes in size.

### FIELD

The present disclosure relates to CpG oligodeoxynucleotides (ODN), and to methods for employing such CpG ODNs as immunostimulants of the fish immune system.

### BACKGROUND

Controlling infectious diseases, caused by different pathogens in aquaculture farms is highly challenging. Diseases outbreak in a short period of time and can cause tremendous economic loss to growers. Antibiotics and chemicals are effective tools against bacterial and parasitical infections, but improper use can cause accumulation of these substances in the environment, accumulation of residues in fish flesh, and emergence of drug-resistant pathogen strains.

Biologically strengthening fish immune defense is one of the most promising methods to control infections in aquaculture. It has been previously reported that unmethylated cytosine phosphate-guanine oligodeoxynucleotides (CpG ODNs) have proved their immunostimulatory actions in mammals and fish (Rebl et al., Veterinary Immunology and Immunopathology 134 (2010) 139-150).

CpG oligodeoxynucleotides (or CpG ODNs) are short single-stranded DNA molecules that contain a cytosine triphosphate deoxynucleotide (“C”) followed by a guanine triphosphate deoxynucleotide (“G”). The “p” refers to the phosphodiester link (or bond) between consecutive nucleotides. Specific CpG motifs, DNA fragments from bacteria and viruses, are pathogen-associated molecular patterns (PAMPs), which are unmethylated ODNs containing a CpG motifs. These CpG motifs can be flanked by certain nucleotide sequences that are abundant in bacterial and viral genomes and rare in vertebrate genomes, which allows the immune system to make the distinction between self and non-self-recognition (Bauer S., Wagner H., 2002, Bacterial CpG-DNA Licenses TLR9. In: Beutler B., Wagner H. (eds) Toll-Like Receptor Family Members and Their Ligands. Current Topics in Microbiology and Immunology, vol 270; Medzhitov, 2007, Nature, Vol. 449). Synthetically-administered ODNs containing unmethylated CpG motifs are well known immunostimulants and potential adjuvants for vaccines, as they have shown to cause protection against bacterial and viral infection in vertebrates including fish (Brudeseth et al., 2013, Fish & Shellfish Immunology, 35, 1759-1768; Rebl et al., 2010, Veterinary Immunology and Immunopathology, 134, 139-150).

CpG's are usually classified into 3 classes (A, B and C), which induce different immune responses (Kang and Kim, 2012, Aquaculture 324-325, 39-43; U-taynapun et al., 2016, Fish & Shellfish Immunology, 58, 116-124). Class A CpG ODNs contain a central palindromic phosphodiester CpG motif and phosphorothioated poly G motifs at the 5′ and 3′ ends, which induces secretion of type I interferon (IFN) and the activation of natural killer (NK) cells. Class B CpG ODNs contain one or more CpG motifs, on phosphorothioate (PS) backbones. This class promotes monocyte maturation and B cell activation. Class C CpG ODNs are known to have structural and functional features of both A and B CpG ODN's and promote activation of both humoral and cellular immunity of both the innate and adaptive immune responses (Kang and Kim, 2012, Aquaculture 324-325, 39-43; U-taynapun et al., 2016, Fish & Shellfish Immunology, 58, 116-124). The CpG ODNs work by binding to intracellular receptors which activate different pathways stimulating the immune system, though many questions remain concerning the molecular mechanism in which CpG's ODN induce its potent immune stimulatory effect (Krieg, 2000, Annu. Rev. Immunol., 20709-760). Other classes or categories of CpG ODN based in part on their sequence, secondary structure and biological effect have also been proposed (Vollmer & Krieg, 2009, Advanced Drug Delivery Reviews, 61, 195-204).

To protect fish from infections, fish farmers use vaccines. However, most of the commercially available vaccines are currently vaccines against bacteria, some are against viruses and none are against parasite or fungal infections. Moreover, the vaccines which are used today are designed for protecting a single fish species or used against a specific bacterial serotype and cannot be used for other fish species or against other bacterial serotypes. Salmonids dominate the commercial focus and are the major market for new developments of vaccines, despite their relatively small contribution to the total volume of farmed fish in the world.

There remains a need in fish farms and aquaculture systems for broad-spectrum fish immunostimulants.

### SUMMARY OF THE DISCLOSURE

The present disclosure describes a technology, i.e. compounds and methods, for use as stimulants of the immune system in fish.

More specifically, provided herein are molecules and compositions comprising CpG ODN sequences which were found to increase the level of key immunological factors in fish, such as IgM, IgD, TLR9, TNFα, TCRα, TCRβ, TCRγ, and TNF.

Further provided herein are methods for initiating and promoting innate and adaptive immunity in fish, and initiating, promoting and increasing an immune response against an antigen in fish.

The present disclosure provides, in one aspect, an immunogenic molecule, comprising an oligonucleotide sequence selected from the group consisting of the oligonucleotide sequence set forth in any one of SEQ ID NO: 2 to SEQ ID NO: 7 and SEQ ID NO: 9, and an immunogenic fragment thereof.

In certain embodiments, the immunogenic comprises an oligonucleotide sequence selected from the group consisting of the oligonucleotide sequence set forth in any one of SEQ ID NO: 3 to SEQ ID NO: 6 and SEQ ID NO: 9.

In certain embodiments, the immunogenic comprises an oligonucleotide sequence selected from the group consisting of the oligonucleotide sequence set forth in any one of SEQ ID NO: 2 to SEQ ID NO: 4, SEQ ID NO: 6, SEQ ID NO: 7 and SEQ ID NO: 9.

In certain embodiments, the immunogenic comprises the oligonucleotide sequence set forth in SEQ ID NO: 2. In certain embodiments, the immunogenic comprises the oligonucleotide sequence set forth in SEQ ID NO: 3. In certain embodiments, the immunogenic comprises the oligonucleotide sequence set forth in SEQ ID NO: 4. In certain embodiments, the immunogenic comprises the oligonucleotide sequence set forth in SEQ ID NO: 5. In certain embodiments, the immunogenic comprises the oligonucleotide sequence set forth in SEQ ID NO: 6. In certain embodiments, the immunogenic comprises the oligonucleotide sequence set forth in SEQ ID NO: 7. In certain embodiments, the immunogenic comprises the oligonucleotide sequence set forth in SEQ ID NO: 9.

In certain embodiments, the oligonucleotide sequence is unmethylated. In certain embodiments, the immunogenic molecule further comprises an additional and unmethylated oligonucleotide sequence. In certain embodiments, the immunogenic molecule further comprises an additional and partly methylated oligonucleotide sequence. In certain embodiments, the immunogenic molecule further comprises an additional and fully methylated oligonucleotide sequence.

In certain embodiments, the immunogenic molecule comprises a phosphodiester link (or bond) between two consecutive CG nucleotides. In certain embodiments, the immunogenic molecule comprises a phosphodiester link between every two consecutive CG nucleotides.

In certain embodiments, the immunogenic molecule comprises a phosphorothioate link between two consecutive CG nucleotides. In certain embodiments, the immunogenic molecule comprises a phosphorothioate link between all two consecutive CG nucleotides. In certain embodiments, the immunogenic molecule comprises a phosphorothioate link between every two consecutive CG nucleotides.

In certain embodiments, the immunogenic molecule comprises a phosphodiester link between two consecutive CG nucleotides and a phosphorothioate link between two other consecutive CG nucleotides. In certain embodiments, the immunogenic molecule comprises a phosphodiester link between two consecutive CG nucleotides and a phosphorothioate link between two consecutive CG nucleotides in every two consecutive CG nucleotides.

In certain embodiments, the phosphorothioate link includes an organic phosphorothioate molecule.

In certain embodiments, the phosphorothioate link includes an inorganic phosphorothioate molecule. In certain embodiments, the inorganic phosphorothioate molecule is a mono-thiophosphate molecule, a di-thiophosphate molecule, a tri-thiophosphate molecule, a tetra-thiophosphate molecule, or a binary thiophosphate/polyphosphate molecule.

In certain embodiments, the immunogenic molecule comprises 10 to 50 nucleotides. In certain embodiments, the immunogenic molecule comprises 20 to 30 nucleotides. In certain embodiments, the immunogenic molecule comprises 22 to 25 nucleotides.

The present disclosure further provides, in another aspect, a composition comprising at least one immunogenic molecule, the immunogenic molecule comprising an oligonucleotide sequence selected from the group consisting of the oligonucleotide sequence set forth in any one of SEQ ID NO: 2 to SEQ ID NO: 7 and SEQ ID NO: 9, and an immunogenic fragment thereof.

In certain embodiments, the composition is a nucleic acid construct.

In certain embodiments, the composition is a fish growing composition used to maintain or grow live fish.

In certain embodiments, the composition is a fish feeding composition used to feed live fish.

The present disclosure further provides, in another aspect, a method of initiating or promoting innate or adaptive immunity in fish, comprising the step of administering at least one immunogenic molecule to the fish, the immunogenic molecule comprising an oligonucleotide sequence selected from the group consisting of the oligonucleotide sequence set forth in any one of SEQ ID NO: 2 to SEQ ID NO: 7 and SEQ ID NO: 9, and an immunogenic fragment thereof.

In certain embodiments, administering the immunogenic molecule to the fish comprises feeding the immunogenic molecule to the fish. In certain embodiments, administering the immunogenic molecule to the fish comprises adding the immunogenic molecule to the medium of the fish. In certain embodiments, administering the immunogenic molecule to the fish comprises contacting the immunogenic molecule with the fish.

The present disclosure further provides, in another aspect, a method for increasing the total IgM level in a fish, comprising the step of administering at least one immunogenic molecule to the fish, the immunogenic molecule comprising an oligonucleotide sequence selected from the group consisting of the oligonucleotide sequence set forth in any one of SEQ ID NO: 2 to SEQ ID NO: 7 and SEQ ID NO: 9, and an immunogenic fragment thereof.

In certain embodiments, the method is for increasing the total IgM level in the spleen, in the head kidney, or in the skin, of the fish.

The present disclosure further provides, in another aspect, a method for increasing the spleen level of IgD, IgM, TNFα, and/or TLR9, in a fish, comprising the step of administering at least one immunogenic molecule to the fish, the immunogenic molecule comprising an oligonucleotide sequence selected from the group consisting of oligonucleotide sequences set forth in any one of SEQ ID NO: 3, SEQ ID NO: 4, SEQ ID NO: 6, and SEQ ID NO: 7.

The present disclosure further provides, in another aspect, a method for increasing the head kidney level of TNFα, TCRβ, IgM, TCRα, TCRγ, TLR9, and/or IgD, in a fish, comprising the step of administering at least one immunogenic molecule to the fish, the immunogenic molecule comprising an oligonucleotide sequence selected from the group consisting of oligonucleotide sequences set forth in any one of SEQ ID NO: 3, SEQ ID NO: 4, SEQ ID NO: 6, and SEQ ID NO: 7.

The present disclosure further provides, in another aspect, a method for increasing the skin level of IgM, IgD, TNFα, TCRα, TCRγ, TLR9, and/or TCRβ, in a fish, comprising the step of administering at least one immunogenic molecule to the fish, the immunogenic molecule comprising an oligonucleotide sequence selected from the group consisting of oligonucleotide sequences set forth in any one of SEQ ID NO: 3, SEQ ID NO: 4, SEQ ID NO: 6, and SEQ ID NO: 7.

In certain embodiments, the administration is repeated at least once every 12 to 36 hours. In certain embodiments, the administration is repeated at least once every 24 hours.

In certain embodiments, the immunogenic molecule comprises the oligonucleotide sequences set forth in any one of SEQ ID NO: 3 to SEQ ID NO: 6 and SEQ ID NO: 9, and an immunogenic fragment thereof.

In certain embodiments, the administration is repeated at least once every 44 to 68 hours. In certain embodiments, the administration is repeated at least once every 56 hours.

In certain embodiments, the immunogenic molecule comprises the oligonucleotide sequences set forth in any one of SEQ ID NO: 2 to SEQ ID NO: 4, SEQ ID NO: 6, SEQ ID NO: 7 and SEQ ID NO: 9, and an immunogenic fragment thereof.

The present disclosure further provides, in another aspect, a method of initiating, promoting or increasing an immune response against an antigen in fish, comprising the step of administering to the fish: (a) at least one immunogenic molecule, comprising an oligonucleotide sequence set forth in any one of SEQ ID NO: 2 to SEQ ID NO: 7 and SEQ ID NO: 9, and an immunogenic fragment thereof, and (b) an antigen.

In certain embodiments, the antigen is derived from or is associated with a bacterium which is pathogenic to fish. In certain embodiments, the antigen is derived from or is associated with a virus which is pathogenic to fish. In certain embodiments, the antigen is derived from or is associated with a parasite which is pathogenic to fish.

In certain embodiments, the antigen is a heat-killed bacterium. In certain embodiments, the bacterium is a gram-negative bacterium. In certain embodiments, the bacterium is a marine bacterium. In certain embodiments, the bacterium is Vibrio harveyi.

In certain embodiments, the fish is from a genus selected from the group consisting of Sparus, Abramis, Acanthopagrus, Argyrops, Blicca, Brama, Chilotilapia, Dicentrarchus, Oncorhynchus, Etelis, Lepomis, Gymnocranius, Lethrinus, Nemipterus, Pharyngochromis, Rhabdosargus, and Scolopsis.

In certain embodiments, the fish belongs to the class Actinopterygii. In certain embodiments, the fish belongs to the order Perciformes. In certain embodiments, the fish is Sea bream (Sparus aurata). In certain embodiments, the fish is Seabass (Dicentrarchus labrax). In certain embodiments, the fish expresses Toll-Like Receptor 9 (TLR9).

In certain embodiments, the immune cells of the fish express TLR9. In certain embodiments, the immune cells are selected from the group consisting of undifferentiated monocytes, macrophages and dendritic cells.

### BRIEF DESCRIPTION OF THE DRAWINGS

FIG. 1 shows the total serum IgM levels (by ELISA analysis, in OD, minus OD of PBS (background)) in fish treated with certain embodiments of the CpG ODNs provided herein, 24 hours and 56 hours post injection.

FIG. 2 shows the total serum IgM levels (by ELISA analysis, in OD) in fish treated with certain embodiments of the CpG ODNs provided herein, 24 hours, 72 hours, 7 days and 14 days post injection.

FIGS. 3A-3B illustrate the hypothetical internal palindrome sequences (shaded) and internal GC dinucleotides (gray) of certain embodiments of the CpG ODNs provided herein.

FIGS. 4A-4C show the nano string analysis for evaluating the up/down regulation of specific Sparus aurata immune genes from spleen (FIG. 4A), head kidney (FIG. 4B) and skin (FIG. 4C) biopsy samples. OD shown is the OD measured minus OD of the PBS-injected control.

FIG. 5 shows total IgM over time in S. aurata injected with 4 different CpGs dosage. Mean±SEM displayed as Fold induction, normalized to calibrator sample minus control fish IgM levels. Carves represent: 2.5, 5, 10, 15, 20 μg CpG injected per fish, PBS-control fish. “a” indicates significant differences (P<0.05), analyzed by one-way ANOVA at each time point (n=5).

FIG. 6 shows total IgM in S. aurata injected with 4 different CpG dosages. Mean±SEM displayed as Fold induction, normalized to calibrator sample minus control fish IgM levels. Bars represent: 2.5, 5, 10, 15, 20 μg of CpG injected per fish, T0—fish IgM level pre-injection. “a” indicates significant differences (P<0.05), analyzed by one-way ANOVA on each time point (n=5).

FIG. 7 shows dosage dependent total IgM levels over time in S. aurata injected with PBS. Mean±SEM displayed as Fold induction, normalized to calibrator sample. Bars represent: 3, 7, 14, 30, and 60 days post injection (n=5).

FIG. 8 shows the total serum IgM levels (by ELISA analysis, in OD, minus OD of PBS (background)) over time in Dicentrarchus labrax treated with the CpG ODNs provided herein. Bars represent: 3, 14 and 30 days post injection. T0—fish IgM level pre-injection. (n=27, for each treatment) showing the base line before any treatment of the fish (IgM level prior to the injection of CpGs).

FIG. 9 shows the fold induction of specific antibodies against Vibrio harveyi in fish serum. CPG—fish vaccinated with a mix of the 4 main CpGs (Cc, Cd, Cf and Cg) together with heat killed Gram-negative bacteria (Vibrio harveyi); VH—fish injected with heat killed Gram-negative bacteria (Vibrio harveyi) mixed with oil adjuvant; PBS—control group injected with PBS mixed with oil adjuvant. T0—before first vaccine and T1-3 weeks after booster injection (6 weeks after initial vaccine) (n=6, for each treatment). Analyzed by one-way ANOVA (sig<0.001, df=5), Tukey Post-Hok

### DETAILED DESCRIPTION OF THE DISCLOSURE

Fish diseases are a major source of depreciation in the aquaculture industry around the world. The main reasons for disease outbreaks are due to rearing conditions, quality of the water and the density of the fish in the farm (on land and in sea cages). The main tools for dealing with these problems are based on prevention by vaccination or treatment with antibiotics and various other chemicals after disease outbreak. Commercial vaccines provide specific immunity against a small number of pathogens for a small number of fish species. The use of antibiotics and various chemicals which are used to treat bacterial or parasitic infections lead to environmental problems, to their accumulation as residual material in fish meat and to the encouragement of resistant pathogenic species.

Strengthening the self-defense mechanism of fish is one of the most promising methods for controlling and regulating the spread of diseases in fish. By using specifically designed DNA fragment motifs of cytosine-phosphate-guanine oligodeoxynucleotides (CpG), the immune system of fish is triggered to stimulate an immune response.

The present disclosure provides an immunostimulant product for fish. The product is based on CpG oligodeoxynucleotides (ODN) which protect fish against a wide range of pathogens and enhance overall fish health. The product is an asset for the marine agriculture market which constantly faces disease and epidemiological challenges. The product can further be used as a specific immune enhancer (adjuvant), combined with an antigen.

As would be understood to a person in the art, the term “CpG ODN” or the term “CpoG” or the term “CpsG” generally refers to an oligodeoxynucleotide comprising at least one C-G motif.

The present disclosure provides, in one aspect, a molecule comprising an oligonucleotide sequence selected from the group consisting of the oligonucleotide sequence set forth in any one of SEQ ID NO: 2 to SEQ ID NO: 7 and SEQ ID NO: 9, and fragments thereof.

In certain embodiments, the molecule is an immunogenic molecule. In certain embodiments, the fragment of the molecule is an immunogenic fragment. As a person in the field would appreciate, the term “immunogenic molecule” as used herein encompasses molecules which provoke an immune response in the body of an animal, including but not limited to the body of a fish. The immune response may be a humoral and/or cell-mediated immune response.

In certain embodiments, the molecule comprises an oligonucleotide sequence selected from the group consisting of the oligonucleotide sequence set forth in any one of SEQ ID NO: 3 to SEQ ID NO: 6 and SEQ ID NO: 9.

In certain embodiments, the molecule comprises an oligonucleotide sequence selected from the group consisting of the oligonucleotide sequence set forth in any one of SEQ ID NO: 2 to SEQ ID NO: 4, SEQ ID NO: 6, SEQ ID NO: 7 and SEQ ID NO: 9.

In certain embodiments, the molecule comprises the oligonucleotide sequence set forth in SEQ ID NO: 2. In certain embodiments, the molecule comprises the oligonucleotide sequence set forth in SEQ ID NO: 3. In certain embodiments, the molecule comprises the oligonucleotide sequence set forth in SEQ ID NO: 4. In certain embodiments, the molecule comprises the oligonucleotide sequence set forth in SEQ ID NO: 5. In certain embodiments, the molecule comprises the oligonucleotide sequence set forth in SEQ ID NO: 6. In certain embodiments, the molecule comprises the oligonucleotide sequence set forth in SEQ ID NO: 7. In certain embodiments, the molecule comprises the oligonucleotide sequence set forth in SEQ ID NO: 9.

In certain embodiments, the molecule consists of the oligonucleotide sequence set forth in SEQ ID NO: 2. In certain embodiments, the molecule consists of the oligonucleotide sequence set forth in SEQ ID NO: 3. In certain embodiments, the molecule consists of the oligonucleotide sequence set forth in SEQ ID NO: 4. In certain embodiments, the molecule consists of the oligonucleotide sequence set forth in SEQ ID NO: 5. In certain embodiments, the molecule consists of the oligonucleotide sequence set forth in SEQ ID NO: 6. In certain embodiments, the molecule consists of the oligonucleotide sequence set forth in SEQ ID NO: 7. In certain embodiments, the molecule consists of the oligonucleotide sequence set forth in SEQ ID NO: 9.

In certain embodiments, the oligonucleotide sequence is unmethylated. As a person of the art would appreciate, the term “DNA methylation” refers to the addition of a methyl group to at least one of two of DNA's four bases, cytosine and adenine.

In certain embodiments, the molecule further comprises an additional and unmethylated oligonucleotide sequence. In certain embodiments, the molecule further comprises an additional and partly methylated oligonucleotide sequence. As a person of the art would appreciate, the term “partial methylation” refers to the addition of a methyl group to part of, at least one but not to all, cytosines and/or adenines in an oligonucleotide sequence.

In certain embodiments, the molecule further comprises an additional and fully methylated oligonucleotide sequence. As a person of the art would appreciate, the term “full methylation” refers to the addition of a methyl group to all of the cytosines and adenines in an oligonucleotide sequence.

In certain embodiments, the molecule comprises a phosphodiester link (or bond) between two consecutive CG nucleotides. As a person of the art would appreciate, the phrase “phosphodiester link between two consecutive CG nucleotides” as used herein refers to a link similar to the link found in the backbone of natural DNA molecules. In certain embodiments, part of the molecule comprises a CpG backbone. In certain embodiments, the molecule comprises a phosphodiester link between every two consecutive CG nucleotides. In certain embodiments, the molecule comprises a CpG backbone. In certain embodiments, a CpG backbone comprises a PO₄ molecule between two nucleotide sugar moieties. In certain embodiments, a CpG backbone comprises a PO₄⁻ molecule between two nucleotide sugar moieties.

In certain embodiments, the molecule comprises a phosphorothioate link between two consecutive CG nucleotides. As a person of the art would appreciate, the phrase “phosphorothioate link between two consecutive CG nucleotides” as used herein refers to a CpG backbone comprising a SPO₃ molecule between two nucleotide sugar moieties. In certain embodiments, the molecule comprises a phosphorothioate link between all two consecutive CG nucleotides. In certain embodiments, the molecule comprises a phosphorothioate link between every two consecutive CG nucleotides. In certain embodiments, the molecule comprises a CpsG backbone. In certain embodiments, the phosphorothioate molecule is SPO₃. In certain embodiments, the phosphorothioate molecule is SPO₃.

In certain embodiments, the molecule comprises a phosphodiester link between two consecutive CG nucleotides and a phosphorothioate link between two other consecutive CG nucleotides. In certain embodiments, part of the molecule comprises a CpG backbone and another part the molecule comprises a CpsG backbone.

In certain embodiments, the molecule comprises a phosphodiester link between two consecutive CG nucleotides and a phosphorothioate link between two consecutive CG nucleotides in all two consecutive CG nucleotides. In certain embodiments, the molecule comprises a phosphodiester link between two consecutive CG nucleotides and a phosphorothioate link between two consecutive CG nucleotides in every two consecutive CG nucleotides. In certain embodiments, the molecule comprises a mixture of CpG and CpsG backbones.

In certain embodiments, the phosphorothioate link includes an organic phosphorothioate molecule.

In certain embodiments, the phosphorothioate link includes an inorganic phosphorothioate molecule. In certain embodiments, the inorganic phosphorothioate molecule is a mono-thiophosphate molecule, a di-thiophosphate molecule, a tri-thiophosphate molecule, a tetra-thiophosphate molecule, or a binary thiophosphate/polyphosphate molecule. In certain embodiments, the inorganic phosphorothioate molecule is a mono-thiophosphate molecule. In certain embodiments, the inorganic phosphorothioate molecule is a di-thiophosphate molecule. In certain embodiments, the inorganic phosphorothioate molecule is a tri-thiophosphate molecule. In certain embodiments, the inorganic phosphorothioate molecule is a tetra-thiophosphate molecule. In certain embodiments, the inorganic phosphorothioate molecule is a binary thiophosphate/polyphosphate molecule.

In certain embodiments, the molecule comprises at least 22 nucleotides. In certain embodiments, the molecule comprises at least 25 nucleotides. In certain embodiments, the molecule comprises at least 30 nucleotides. In certain embodiments, the molecule comprises at least 35 nucleotides. In certain embodiments, the molecule comprises at least 40 nucleotides. In certain embodiments, the molecule comprises at least 45 nucleotides. In certain embodiments, the molecule comprises at least 50 nucleotides.

In certain embodiments, the molecule comprises 22 to 220 nucleotides. In certain embodiments, the molecule comprises 25 to 220 nucleotides. In certain embodiments, the molecule comprises 22 to 150 nucleotides. In certain embodiments, the molecule comprises 25 to 150 nucleotides. In certain embodiments, the molecule comprises 22 to 100 nucleotides. In certain embodiments, the molecule comprises 25 to 100 nucleotides. In certain embodiments, the molecule comprises 22 to 50 nucleotides. In certain embodiments, the molecule comprises 25 to 50 nucleotides. In certain embodiments, the molecule comprises 6 to 40 nucleotides. In certain embodiments, the molecule comprises 20 to 30 nucleotides. In certain embodiments, the molecule comprises 22 to 25 nucleotides.

The term “fragment” as used herein refers to at least 6 consecutive nucleotides of a corresponding nucleotide sequence. As a person of the field would appreciate, an immunogenic fragment of any immunogenic sequence would be shorter than the corresponding immunogenic sequence but would retain at least 50% of the immunogenicity of the corresponding immunogenic sequence.

In certain embodiments, an immunogenic fragment comprises at least 6 consecutive nucleotides of a corresponding nucleotide sequence. In certain embodiments, an immunogenic comprises at least 10 consecutive nucleotides of a corresponding nucleotide sequence. In certain embodiments, an immunogenic comprises at least 15 consecutive nucleotides of a corresponding nucleotide sequence. In certain embodiments, an immunogenic comprises at least 20 consecutive nucleotides of a corresponding nucleotide sequence.

In certain embodiments, an immunogenic fragment comprises at least 6 consecutive nucleotides of a corresponding nucleotide sequence, and retains at least 50% of the immunogenicity of the corresponding nucleotide sequence. In certain embodiments, an immunogenic comprises at least 10 consecutive nucleotides of a corresponding nucleotide sequence, and retains at least 50% of the immunogenicity of the corresponding nucleotide sequence. In certain embodiments, an immunogenic comprises at least 15 consecutive nucleotides of a corresponding nucleotide sequence, and retains at least 50% of the immunogenicity of the corresponding nucleotide sequence. In certain embodiments, an immunogenic comprises at least 20 consecutive nucleotides of a corresponding nucleotide sequence, and retains at least 50% of the immunogenicity of the corresponding nucleotide sequence.

In certain embodiments, an immunogenic fragment comprises at least 6 consecutive nucleotides of a corresponding nucleotide sequence, and retains at least 75% of the immunogenicity of the corresponding nucleotide sequence. In certain embodiments, an immunogenic comprises at least 10 consecutive nucleotides of a corresponding nucleotide sequence, and retains at least 75% of the immunogenicity of the corresponding nucleotide sequence. In certain embodiments, an immunogenic comprises at least 15 consecutive nucleotides of a corresponding nucleotide sequence, and retains at least 75% of the immunogenicity of the corresponding nucleotide sequence. In certain embodiments, an immunogenic comprises at least 20 consecutive nucleotides of a corresponding nucleotide sequence, and retains at least 75% of the immunogenicity of the corresponding nucleotide sequence.

In certain embodiments, an immunogenic fragment comprises at least 6 consecutive nucleotides of a corresponding nucleotide sequence, and retains at least 90% of the immunogenicity of the corresponding nucleotide sequence. In certain embodiments, an immunogenic comprises at least 10 consecutive nucleotides of a corresponding nucleotide sequence, and retains at least 90% of the immunogenicity of the corresponding nucleotide sequence. In certain embodiments, an immunogenic comprises at least 15 consecutive nucleotides of a corresponding nucleotide sequence, and retains at least 90% of the immunogenicity of the corresponding nucleotide sequence. In certain embodiments, an immunogenic comprises at least 20 consecutive nucleotides of a corresponding nucleotide sequence, and retains at least 90% of the immunogenicity of the corresponding nucleotide sequence.

The present disclosure further provides, in another aspect, a composition comprising a molecule, the molecule comprising an oligonucleotide sequence selected from the group consisting of the oligonucleotide sequence set forth in any one of SEQ ID NO: 2 to SEQ ID NO: 7 and SEQ ID NO: 9, and a fragment thereof. In certain embodiments, the composition comprises at least one molecule, the molecule comprising an oligonucleotide sequence selected from the group consisting of the oligonucleotide sequence set forth in any one of SEQ ID NO: 2 to SEQ ID NO: 7 and SEQ ID NO: 9, and a fragment thereof.

In certain embodiments, the composition comprises at least one molecule. In certain embodiments, the composition comprises one molecule. In certain embodiments, the composition comprises two molecules. In certain embodiments, the composition comprises 3 molecules. In certain embodiments, the composition comprises 4 molecules. In certain embodiments, the composition comprises 5 molecules. In certain embodiments, the composition comprises 6 molecules. In certain embodiments, the composition comprises 7 molecules.

In certain embodiments, the molecule is an immunogenic molecule. In certain embodiments, the fragment of the molecule is an immunogenic fragment.

In certain embodiments, the composition is a nucleic acid construct. In certain embodiments, the composition is a fish growing composition used to maintain or grow live fish. In certain embodiments, the fish growing composition used to maintain or grow live fish is saltwater. In certain embodiments, the fish growing composition used to maintain or grow live fish is sea water.

In certain embodiments, the composition is a fish feeding composition used to feed live fish. In certain embodiments, the fish feeding composition used to feed live fish is Sea bream fish food.

In certain embodiments, the composition comprises at least about 2.5 μg of the oligonucleotide sequence. In certain embodiments, the composition comprises at least about 5 μg of the oligonucleotide sequence. In certain embodiments, the composition comprises at least about 10 μg of the oligonucleotide sequence. In certain embodiments, the composition comprises at least about 15 μg of the oligonucleotide sequence. In certain embodiments, the composition comprises at least about 20 μg of the oligonucleotide sequence.

In certain embodiments, the composition comprises about 2.5 to about 20 μg of the oligonucleotide sequence. In certain embodiments, the composition comprises about 5 to about 20 μg of the oligonucleotide sequence. In certain embodiments, the composition comprises about 10 to about 20 μg of the oligonucleotide sequence. In certain embodiments, the composition comprises about 15 to about 20 μg of the oligonucleotide sequence.

In certain embodiments, the composition comprises micro-encapsulated molecule comprising an oligonucleotide sequence selected from the group consisting of the oligonucleotide sequence set forth in any one of SEQ ID NO: 2 to SEQ ID NO: 7 and SEQ ID NO: 9, and a fragment thereof.

The present disclosure further provides, in another aspect, a method of initiating or promoting innate or adaptive immunity in fish, comprising the step of administering at least one immunogenic molecule to the fish, the immunogenic molecule comprising an oligonucleotide sequence selected from the group consisting of the oligonucleotide sequence set forth in any one of SEQ ID NO: 2 to SEQ ID NO: 7 and SEQ ID NO: 9, and an immunogenic fragment thereof.

In certain embodiments, the step of administering the immunogenic molecule to the fish comprises feeding the immunogenic molecule to the fish. In certain embodiments, the step of administering the immunogenic molecule to the fish comprises adding the immunogenic molecule to the medium of the fish. In certain embodiments, the step of administering the immunogenic molecule to the fish comprises contacting the immunogenic molecule with the fish.

The present disclosure further provides, in another aspect, a method for increasing the total IgM level in a fish, comprising the step of administering at least one immunogenic molecule to the fish, the immunogenic molecule comprising an oligonucleotide sequence selected from the group consisting of the oligonucleotide sequence set forth in any one of SEQ ID NO: 2 to SEQ ID NO: 7 and SEQ ID NO: 9, and an immunogenic fragment thereof.

In certain embodiments, the method is for increasing the total IgM level in the spleen, in the head kidney, or in the skin, of the fish. In certain embodiments, the method is for increasing the total IgM level in the spleen of the fish. In certain embodiments, the method is for increasing the total IgM level in the head kidney of the fish. In certain embodiments, the method is for increasing the total IgM level in the skin of the fish.

The present disclosure further provides, in another aspect, a method for increasing the spleen level of IgD, IgM, TNFα, and/or TLR9, in a fish, comprising the step of administering at least one immunogenic molecule to the fish, the immunogenic molecule comprising an oligonucleotide sequence selected from the group consisting of oligonucleotide sequences set forth in any one of SEQ ID NO: 3, SEQ ID NO: 4, SEQ ID NO: 6, and SEQ ID NO: 7.

In certain embodiments, the method is for increasing the spleen level of IgD, comprising administrating an oligonucleotide sequence comprising the oligonucleotide sequences set forth in SEQ ID NO: 3. In certain embodiments, the method is for increasing the spleen level of IgM, comprising administrating an oligonucleotide sequence comprising the oligonucleotide sequences set forth in SEQ ID NO: 3. In certain embodiments, the method is for increasing the spleen level of TNFα, comprising administrating an oligonucleotide sequence comprising the oligonucleotide sequences set forth in SEQ ID NO: 3. In certain embodiments, the method is for increasing the spleen level of TLR9, comprising administrating an oligonucleotide sequence comprising the oligonucleotide sequences set forth in SEQ ID NO: 3.

In certain embodiments, the method is for increasing the spleen level of IgD, comprising administrating an oligonucleotide sequence comprising the oligonucleotide sequences set forth in SEQ ID NO: 4. In certain embodiments, the method is for increasing the spleen level of IgM, comprising administrating an oligonucleotide sequence comprising the oligonucleotide sequences set forth in SEQ ID NO: 4. In certain embodiments, the method is for increasing the spleen level of TNFα, comprising administrating an oligonucleotide sequence comprising the oligonucleotide sequences set forth in SEQ ID NO: 4. In certain embodiments, the method is for increasing the spleen level of TLR9, comprising administrating an oligonucleotide sequence comprising the oligonucleotide sequences set forth in SEQ ID NO: 4.

In certain embodiments, the method is for increasing the spleen level of IgD, comprising administrating an oligonucleotide sequence comprising the oligonucleotide sequences set forth in SEQ ID NO: 6. In certain embodiments, the method is for increasing the spleen level of IgM, comprising administrating an oligonucleotide sequence comprising the oligonucleotide sequences set forth in SEQ ID NO: 6. In certain embodiments, the method is for increasing the spleen level of TNFα, comprising administrating an oligonucleotide sequence comprising the oligonucleotide sequences set forth in SEQ ID NO: 6. In certain embodiments, the method is for increasing the spleen level of TLR9, comprising administrating an oligonucleotide sequence comprising the oligonucleotide sequences set forth in SEQ ID NO: 6.

In certain embodiments, the method is for increasing the spleen level of IgD, comprising administrating an oligonucleotide sequence comprising the oligonucleotide sequences set forth in SEQ ID NO: 7. In certain embodiments, the method is for increasing the spleen level of IgM, comprising administrating an oligonucleotide sequence comprising the oligonucleotide sequences set forth in SEQ ID NO: 7. In certain embodiments, the method is for increasing the spleen level of TNFα, comprising administrating an oligonucleotide sequence comprising the oligonucleotide sequences set forth in SEQ ID NO: 7. In certain embodiments, the method is for increasing the spleen level of TLR9, comprising administrating an oligonucleotide sequence comprising the oligonucleotide sequences set forth in SEQ ID NO: 7.

The present disclosure further provides, in another aspect, a method for increasing the head kidney level of TNFα, TCRβ, IgM, TCRα, TCRγ, TLR9, and/or IgD, in a fish, comprising the step of administering at least one immunogenic molecule to the fish, the immunogenic molecule comprising an oligonucleotide sequence selected from the group consisting of oligonucleotide sequences set forth in any one of SEQ ID NO: 3, SEQ ID NO: 4, SEQ ID NO: 6, and SEQ ID NO: 7.

In certain embodiments, the method is for increasing the spleen level of IgD, comprising administrating an oligonucleotide sequence comprising the oligonucleotide sequences set forth in SEQ ID NO: 3. In certain embodiments, the method is for increasing the spleen level of IgM, comprising administrating an oligonucleotide sequence comprising the oligonucleotide sequences set forth in SEQ ID NO: 3. In certain embodiments, the method is for increasing the spleen level of TNFα, comprising administrating an oligonucleotide sequence comprising the oligonucleotide sequences set forth in SEQ ID NO: 3. In certain embodiments, the method is for increasing the spleen level of TLR9, comprising administrating an oligonucleotide sequence comprising the oligonucleotide sequences set forth in SEQ ID NO: 3. In certain embodiments, the method is for increasing the spleen level of TCRβ, comprising administrating an oligonucleotide sequence comprising the oligonucleotide sequences set forth in SEQ ID NO: 3. In certain embodiments, the method is for increasing the spleen level of TCRα, comprising administrating an oligonucleotide sequence comprising the oligonucleotide sequences set forth in SEQ ID NO: 3. In certain embodiments, the method is for increasing the spleen level of TCRγ, comprising administrating an oligonucleotide sequence comprising the oligonucleotide sequences set forth in SEQ ID NO: 3.

In certain embodiments, the method is for increasing the spleen level of IgD, comprising administrating an oligonucleotide sequence comprising the oligonucleotide sequences set forth in SEQ ID NO: 4. In certain embodiments, the method is for increasing the spleen level of IgM, comprising administrating an oligonucleotide sequence comprising the oligonucleotide sequences set forth in SEQ ID NO: 4. In certain embodiments, the method is for increasing the spleen level of TNFα, comprising administrating an oligonucleotide sequence comprising the oligonucleotide sequences set forth in SEQ ID NO: 4. In certain embodiments, the method is for increasing the spleen level of TLR9, comprising administrating an oligonucleotide sequence comprising the oligonucleotide sequences set forth in SEQ ID NO: 4. In certain embodiments, the method is for increasing the spleen level of TCRβ, comprising administrating an oligonucleotide sequence comprising the oligonucleotide sequences set forth in SEQ ID NO: 4. In certain embodiments, the method is for increasing the spleen level of TCRα, comprising administrating an oligonucleotide sequence comprising the oligonucleotide sequences set forth in SEQ ID NO: 4. In certain embodiments, the method is for increasing the spleen level of TCRγ, comprising administrating an oligonucleotide sequence comprising the oligonucleotide sequences set forth in SEQ ID NO: 4.

In certain embodiments, the method is for increasing the spleen level of IgD, comprising administrating an oligonucleotide sequence comprising the oligonucleotide sequences set forth in SEQ ID NO: 6. In certain embodiments, the method is for increasing the spleen level of IgM, comprising administrating an oligonucleotide sequence comprising the oligonucleotide sequences set forth in SEQ ID NO: 6. In certain embodiments, the method is for increasing the spleen level of TNFα, comprising administrating an oligonucleotide sequence comprising the oligonucleotide sequences set forth in SEQ ID NO: 6. In certain embodiments, the method is for increasing the spleen level of TLR9, comprising administrating an oligonucleotide sequence comprising the oligonucleotide sequences set forth in SEQ ID NO: 6. In certain embodiments, the method is for increasing the spleen level of TCRβ, comprising administrating an oligonucleotide sequence comprising the oligonucleotide sequences set forth in SEQ ID NO: 6. In certain embodiments, the method is for increasing the spleen level of TCRα, comprising administrating an oligonucleotide sequence comprising the oligonucleotide sequences set forth in SEQ ID NO: 6. In certain embodiments, the method is for increasing the spleen level of TCRγ, comprising administrating an oligonucleotide sequence comprising the oligonucleotide sequences set forth in SEQ ID NO: 6.

In certain embodiments, the method is for increasing the spleen level of IgD, comprising administrating an oligonucleotide sequence comprising the oligonucleotide sequences set forth in SEQ ID NO: 7. In certain embodiments, the method is for increasing the spleen level of IgM, comprising administrating an oligonucleotide sequence comprising the oligonucleotide sequences set forth in SEQ ID NO: 7. In certain embodiments, the method is for increasing the spleen level of TNFα, comprising administrating an oligonucleotide sequence comprising the oligonucleotide sequences set forth in SEQ ID NO: 7. In certain embodiments, the method is for increasing the spleen level of TLR9, comprising administrating an oligonucleotide sequence comprising the oligonucleotide sequences set forth in SEQ ID NO: 7. In certain embodiments, the method is for increasing the spleen level of TCRβ, comprising administrating an oligonucleotide sequence comprising the oligonucleotide sequences set forth in SEQ ID NO: 7. In certain embodiments, the method is for increasing the spleen level of TCRα, comprising administrating an oligonucleotide sequence comprising the oligonucleotide sequences set forth in SEQ ID NO: 7. In certain embodiments, the method is for increasing the spleen level of TCRγ, comprising administrating an oligonucleotide sequence comprising the oligonucleotide sequences set forth in SEQ ID NO: 7.

The present disclosure further provides, in another aspect, a method for increasing the skin level of IgM, IgD, TNFα, TCRα, TCRγ, TLR9, and/or TCRβ, in a fish, comprising the step of administering at least one immunogenic molecule to the fish, the immunogenic molecule comprising an oligonucleotide sequence selected from the group consisting of oligonucleotide sequences set forth in any one of SEQ ID NO: 3, SEQ ID NO: 4, SEQ ID NO: 6, and SEQ ID NO: 7.

In certain embodiments, the method is for increasing the spleen level of IgD, comprising administrating an oligonucleotide sequence comprising the oligonucleotide sequences set forth in SEQ ID NO: 3. In certain embodiments, the method is for increasing the spleen level of IgM, comprising administrating an oligonucleotide sequence comprising the oligonucleotide sequences set forth in SEQ ID NO: 3. In certain embodiments, the method is for increasing the spleen level of TNFα, comprising administrating an oligonucleotide sequence comprising the oligonucleotide sequences set forth in SEQ ID NO: 3. In certain embodiments, the method is for increasing the spleen level of TLR9, comprising administrating an oligonucleotide sequence comprising the oligonucleotide sequences set forth in SEQ ID NO: 3. In certain embodiments, the method is for increasing the spleen level of TCRβ, comprising administrating an oligonucleotide sequence comprising the oligonucleotide sequences set forth in SEQ ID NO: 3. In certain embodiments, the method is for increasing the spleen level of TCRα, comprising administrating an oligonucleotide sequence comprising the oligonucleotide sequences set forth in SEQ ID NO: 3. In certain embodiments, the method is for increasing the spleen level of TCRγ, comprising administrating an oligonucleotide sequence comprising the oligonucleotide sequences set forth in SEQ ID NO: 3.

In certain embodiments, the method is for increasing the spleen level of IgD, comprising administrating an oligonucleotide sequence comprising the oligonucleotide sequences set forth in SEQ ID NO: 4. In certain embodiments, the method is for increasing the spleen level of IgM, comprising administrating an oligonucleotide sequence comprising the oligonucleotide sequences set forth in SEQ ID NO: 4. In certain embodiments, the method is for increasing the spleen level of TNFα, comprising administrating an oligonucleotide sequence comprising the oligonucleotide sequences set forth in SEQ ID NO: 4. In certain embodiments, the method is for increasing the spleen level of TLR9, comprising administrating an oligonucleotide sequence comprising the oligonucleotide sequences set forth in SEQ ID NO: 4. In certain embodiments, the method is for increasing the spleen level of TCRβ, comprising administrating an oligonucleotide sequence comprising the oligonucleotide sequences set forth in SEQ ID NO: 4. In certain embodiments, the method is for increasing the spleen level of TCRα, comprising administrating an oligonucleotide sequence comprising the oligonucleotide sequences set forth in SEQ ID NO: 4. In certain embodiments, the method is for increasing the spleen level of TCRγ, comprising administrating an oligonucleotide sequence comprising the oligonucleotide sequences set forth in SEQ ID NO: 4.

In certain embodiments, the method is for increasing the spleen level of IgD, comprising administrating an oligonucleotide sequence comprising the oligonucleotide sequences set forth in SEQ ID NO: 6. In certain embodiments, the method is for increasing the spleen level of IgM, comprising administrating an oligonucleotide sequence comprising the oligonucleotide sequences set forth in SEQ ID NO: 6. In certain embodiments, the method is for increasing the spleen level of TNFα, comprising administrating an oligonucleotide sequence comprising the oligonucleotide sequences set forth in SEQ ID NO: 6. In certain embodiments, the method is for increasing the spleen level of TLR9, comprising administrating an oligonucleotide sequence comprising the oligonucleotide sequences set forth in SEQ ID NO: 6. In certain embodiments, the method is for increasing the spleen level of TCRβ, comprising administrating an oligonucleotide sequence comprising the oligonucleotide sequences set forth in SEQ ID NO: 6. In certain embodiments, the method is for increasing the spleen level of TCRα, comprising administrating an oligonucleotide sequence comprising the oligonucleotide sequences set forth in SEQ ID NO: 6. In certain embodiments, the method is for increasing the spleen level of TCRγ, comprising administrating an oligonucleotide sequence comprising the oligonucleotide sequences set forth in SEQ ID NO: 6.

In certain embodiments, the method is for increasing the spleen level of IgD, comprising administrating an oligonucleotide sequence comprising the oligonucleotide sequences set forth in SEQ ID NO: 7. In certain embodiments, the method is for increasing the spleen level of IgM, comprising administrating an oligonucleotide sequence comprising the oligonucleotide sequences set forth in SEQ ID NO: 7. In certain embodiments, the method is for increasing the spleen level of TNFα, comprising administrating an oligonucleotide sequence comprising the oligonucleotide sequences set forth in SEQ ID NO: 7. In certain embodiments, the method is for increasing the spleen level of TLR9, comprising administrating an oligonucleotide sequence comprising the oligonucleotide sequences set forth in SEQ ID NO: 7. In certain embodiments, the method is for increasing the spleen level of TCRβ, comprising administrating an oligonucleotide sequence comprising the oligonucleotide sequences set forth in SEQ ID NO: 7. In certain embodiments, the method is for increasing the spleen level of TCRα, comprising administrating an oligonucleotide sequence comprising the oligonucleotide sequences set forth in SEQ ID NO: 7. In certain embodiments, the method is for increasing the spleen level of TCRγ, comprising administrating an oligonucleotide sequence comprising the oligonucleotide sequences set forth in SEQ ID NO: 7.

In certain embodiments, the administration is repeated at least once every 12 to 36 hours. In certain embodiments, the administration is repeated at least once every 24 hours. In certain embodiments, the administration is repeated at least twice every 12 to 36 hours. In certain embodiments, the administration is repeated at least twice every 24 hours. In certain embodiments, the administration is repeated at least trice every 12 to 36 hours. In certain embodiments, the administration is repeated at least trice every 24 hours. In certain embodiments, the immunogenic molecule comprises the oligonucleotide sequences set forth in any one of SEQ ID NO: 3 to SEQ ID NO: 6 and SEQ ID NO: 9, and an immunogenic fragment thereof. In certain embodiments, the immunogenic molecule comprises the oligonucleotide sequences set forth in SEQ ID NO: 3 or an immunogenic fragment thereof. In certain embodiments, the immunogenic molecule comprises the oligonucleotide sequences set forth in SEQ ID NO: 4 or an immunogenic fragment thereof. In certain embodiments, the immunogenic molecule comprises the oligonucleotide sequences set forth in SEQ ID NO: 5 or an immunogenic fragment thereof. In certain embodiments, the immunogenic molecule comprises the oligonucleotide sequences set forth in SEQ ID NO: 6 or an immunogenic fragment thereof. In certain embodiments, the immunogenic molecule comprises the oligonucleotide sequences set forth in SEQ ID NO: 9 or an immunogenic fragment thereof.

In certain embodiments, the administration is repeated at least once every 44 to 68 hours. In certain embodiments, the administration is repeated at least once every 56 hours. In certain embodiments, the administration is repeated at least twice every 44 to 68 hours. In certain embodiments, the administration is repeated at least twice every 56 hours. In certain embodiments, the administration is repeated at least trice every 44 to 68 hours. In certain embodiments, the administration is repeated at least trice every 56 hours. In certain embodiments, the immunogenic molecule comprises the oligonucleotide sequences set forth in any one of SEQ ID NO: 2 to SEQ ID NO: 4, SEQ ID NO: 6, SEQ ID NO: 7 and SEQ ID NO: 9, and an immunogenic fragment thereof. In certain embodiments, the immunogenic molecule comprises the oligonucleotide sequences set forth in SEQ ID NO: 2 or an immunogenic fragment thereof. In certain embodiments, the immunogenic molecule comprises the oligonucleotide sequences set forth in SEQ ID NO: 3 or an immunogenic fragment thereof. In certain embodiments, the immunogenic molecule comprises the oligonucleotide sequences set forth in SEQ ID NO: 4 or an immunogenic fragment thereof. In certain embodiments, the immunogenic molecule comprises the oligonucleotide sequences set forth in SEQ ID NO: 6 or an immunogenic fragment thereof. In certain embodiments, the immunogenic molecule comprises the oligonucleotide sequences set forth in SEQ ID NO: 7 or an immunogenic fragment thereof. In certain embodiments, the immunogenic molecule comprises the oligonucleotide sequences set forth in SEQ ID NO: 9 or an immunogenic fragment thereof.

The present disclosure further provides, in another aspect, a method of initiating, promoting or increasing an immune response against an antigen in fish, comprising the step of administering to the fish: (a) at least one immunogenic molecule, comprising an oligonucleotide sequence set forth in any one of SEQ ID NO: 2 to SEQ ID NO: 7 and SEQ ID NO: 9, and an immunogenic fragment thereof, and (b) an antigen. In certain embodiments, the method comprises administering (a) at least one immunogenic molecule, comprising the oligonucleotide sequence set forth in SEQ ID NO: 2 or an immunogenic fragment thereof, and (b) the antigen. In certain embodiments, the method comprises administering (a) at least one immunogenic molecule, comprising the oligonucleotide sequence set forth in SEQ ID NO: 3 or an immunogenic fragment thereof, and (b) the antigen. In certain embodiments, the method comprises administering (a) at least one immunogenic molecule, comprising the oligonucleotide sequence set forth in SEQ ID NO: 4 or an immunogenic fragment thereof, and (b) the antigen. In certain embodiments, the method comprises administering (a) at least one immunogenic molecule, comprising the oligonucleotide sequence set forth in SEQ ID NO: 5 or an immunogenic fragment thereof, and (b) the antigen. In certain embodiments, the method comprises administering (a) at least one immunogenic molecule, comprising the oligonucleotide sequence set forth in SEQ ID NO: 6 or an immunogenic fragment thereof, and (b) the antigen. In certain embodiments, the method comprises administering (a) at least one immunogenic molecule, comprising the oligonucleotide sequence set forth in SEQ ID NO: 7 or an immunogenic fragment thereof, and (b) the antigen. In certain embodiments, the method comprises administering (a) at least one immunogenic molecule, comprising the oligonucleotide sequence set forth in SEQ ID NO: 9 or an immunogenic fragment thereof, and (b) the antigen.

In certain embodiments, the antigen is derived from or is associated with a virus which is pathogenic to fish. In certain embodiments, the antigen is derived from or is associated with a parasite which is pathogenic to fish. In certain embodiments, the antigen is derived from or is associated with a bacterium which is pathogenic to fish. In certain embodiments, the antigen is a whole bacterium. In certain embodiments, the antigen is a heat-killed bacterium.

In certain embodiments, the bacterium is a gram-negative bacterium. In certain embodiments, the bacterium is a gram-positive bacterium. In certain embodiments, the bacterium is a heat-killed bacterium. In certain embodiments, the bacterium is a marine bacterium. In certain embodiments, the bacterium is a freshwater bacterium.

In certain embodiments, the bacterium is from a Genus selected from the group consisting of Vibrio, Pseudomonas, and Flavobacterium. In certain embodiments, the bacterium is from the Genus Vibrio. In certain embodiments, the bacterium is from the Genus Pseudomonas. In certain embodiments, the bacterium is from the Genus Flavobacterium.

In certain embodiments, the bacterium is from a Class selected from Gammaproteobacteria. or Flavobacteriia. In certain embodiments, the bacterium is from the Class Gammaproteobacteria. In certain embodiments, the bacterium is from the Class Flavobacteriia.

In certain embodiments, the bacterium is from an Order selected from the group consisting of Enterobacterales, Vibrionales, Pseudomonadales, and Flavobacteriales. In certain embodiments, the bacterium is from an Order Enterobacterales. In certain embodiments, the bacterium is from an Order Vibrionales. In certain embodiments, the bacterium is from an Order Pseudomonadales. In certain embodiments, the bacterium is from an Order Flavobacteriales.

In certain embodiments, the bacterium is from a Family selected from the group consisting of Enterobacteriaceae, Vibrionaceae, Pseudomonadaceae, and Flavobacteriaceae. In certain embodiments, the bacterium is from the Family Enterobacteriaceae. In certain embodiments, the bacterium is from the Family Vibrionaceae. In certain embodiments, the bacterium is from the Family Pseudomonadaceae. In certain embodiments, the bacterium is from the Family Flavobacteriaceae.

In certain embodiments, the bacterium is selected from the group consisting of Vibrio harveyi, Vibrio parahaemolyticus, Vibrio alginolyticus, Vibrio anguillarum. and Vibrio owensii. In certain embodiments, the bacterium is Vibrio harveyi. In certain embodiments, the bacterium is Vibrio parahaemolyticus. In certain embodiments, the bacterium is Vibrio alginolyticus. In certain embodiments, the bacterium is Vibrio owensii. In certain embodiments, the bacterium is Vibrio anguillarum

In certain embodiments, the fish is from a Genus selected from the group consisting of Sparus, Abramis, Acanthopagrus, Argyrops, Blicca, Brama, Chilotilapia, Dicentrarchus, Oncorhynchus. Etelis, Lepomis, Gymnocranius, Lethrinus, Nemipterus, Pharyngochromis, Rhabdosargus, and Scolopsis. In certain embodiments, the fish is from the Genus Sparus. In certain embodiments, the fish is from the Genus Abramis. In certain embodiments, the fish is from the Genus Acanthopagrus. In certain embodiments, the fish is from the Genus Argyrops. In certain embodiments, the fish is from the Genus Blicca. In certain embodiments, the fish is from the Genus Brama. In certain embodiments, the fish is from the Genus Chilotilapia. In certain embodiments, the fish is from the Genus Dicentrarchus. In certain embodiments, the fish is from the Genus Oncorhynchus. In certain embodiments, the fish is from the Genus Etelis. In certain embodiments, the fish is from the Genus Lepomis. In certain embodiments, the fish is from the Genus Gymnocranius. In certain embodiments, the fish is from the Genus Lethrinus. In certain embodiments, the fish is from the Genus Nemipterus. In certain embodiments, the fish is from the Genus Pharyngochromis. In certain embodiments, the fish is from the Genus Rhabdosargus. In certain embodiments, the fish is from the Genus Scolopsis.

In certain embodiments, the fish belongs to the Class Actinopterygii. In certain embodiments, the fish belongs to the Order Perciformes. In certain embodiments, the fish belongs to the Family Sparidae. In certain embodiments, the fish is Sea bream (Sparus aurata).

In certain embodiments, the fish belongs to the Family Moronidae. In certain embodiments, the fish is Seabass (Dicentrarchus labrax).

In certain embodiments, the fish expresses Toll-Like Receptor 9 (TLR9). In certain embodiments, the immune cells of the fish expresses TLR9. In certain embodiments, the immune cells are selected from the group consisting of undifferentiated monocytes, macrophages and dendritic cells. In certain embodiments, the immune cells are undifferentiated monocytes. In certain embodiments, the immune cells are macrophages. In certain embodiments, the immune cells are dendritic cells.

In certain embodiments, the method comprises administering at least about 2.5 μg of the oligonucleotide sequence to each fish. In certain embodiments, the method comprises administering at least about 5 μg of the oligonucleotide sequence to each fish. In certain embodiments, the method comprises administering at least about 10 μg of the oligonucleotide sequence to each fish. In certain embodiments, the method comprises administering at least about 15 μg of the oligonucleotide sequence to each fish. In certain embodiments, the method comprises administering at least about 20 μg of the oligonucleotide sequence to each fish.

In certain embodiments, the method comprises administering about 2.5 μg to about 20 μg of the oligonucleotide sequence to each fish. In certain embodiments, the method comprises administering about 5 μg to about 20 μg of the oligonucleotide sequence to each fish. In certain embodiments, the method comprises administering about 10 μg to about 20 μg of the oligonucleotide sequence to each fish. In certain embodiments, the method comprises administering about 15 μg to about 20 μg of the oligonucleotide sequence to each fish.

In certain embodiments, the immunogenic molecule comprises a Class A CpG ODN. In certain embodiments, a Class A CpG ODN comprises a poly-G sequence at the 5′ end. In certain embodiments, a Class A CpG ODN comprises a poly-G sequence at the 3′ end. In certain embodiments, a Class A CpG ODN comprises a poly-G sequence at the 5′ end and at the 3′ end. In certain embodiments, a Class A CpG ODN comprises an internal palindrome sequence. In certain embodiments, the internal palindrome sequence comprises GC dinucleotides. In certain embodiments, the internal palindrome sequence comprises a phosphorothioated (PS) backbone.

In certain embodiments, the immunogenic molecule comprises a Class B CpG ODN. In certain embodiments, a Class B CpG ODN comprises a 6-mer CpG motif 5′-Pu-Py-C-G-Py-Pu-3′. In certain embodiments, a Class B CpG ODN comprises three 6-mer sequences. In certain embodiments, a Class B CpG ODN comprises a full phosphorothioated (PS) backbone. In certain embodiments, a Class B CpG ODN comprises 18 to 28 nucleotides.

In certain embodiments, the immunogenic molecule comprises a Class C CpG ODN. In certain embodiments, a Class C CpG ODN comprises a full phosphorothioated (PS) backbone. In certain embodiments, a Class C CpG ODN comprises an internal palindrome sequence. In certain embodiments, the internal palindrome sequence comprises GC dinucleotides.

In certain embodiments, the immunogenic molecule comprises a Class P CpG ODN. In certain embodiments, a Class P CpG ODN comprises two internal palindrome sequences. In certain embodiments, a Class P CpG ODN comprises a full phosphorothioated (PS) backbone.

In certain embodiments, the immunogenic molecule comprises a Class S CpG ODN. In certain embodiments, a Class S CpG ODN block immune stimulatory effects. In certain embodiments, the immune stimulatory effects are mediated by CpG ODN, pathogens, or self-DNA. In certain embodiments, a Class S CpG ODN inhibits TLR7- and TLR8-mediated responses stimulated by self RNA or viral RNA. In certain embodiments, a Class S CpG ODN inhibits TLR-mediated stimulation induced by DNA- and RNA-containing immune complexes.

In certain embodiments, the immunogenic molecule comprises a Type D CpG ODN. In certain embodiments, a Type D CpG ODN triggers plasmacytoid dendritic cells (pDCs) to produce interferon-α (IFN-α).

In certain embodiments, the immunogenic molecule comprises a Type K CpG ODN. In certain embodiments, a Type K CpG ODN triggers plasmacytoid dendritic cells (pDCs) to mature and secrete TNF-α.

### EXAMPLES

### Example 1—CpG ODN Administration Increases Total IgM Levels in Sea Bream Serum

CpG ODNs—Eight (8) different CpG class C ODNs were designed to induce and stimulate innate and adaptive immunity in fish, labeled Cb to Ci. A known class C CpG ODN, Ca, was also included. A control non-CpG ODN is used as a negative control.

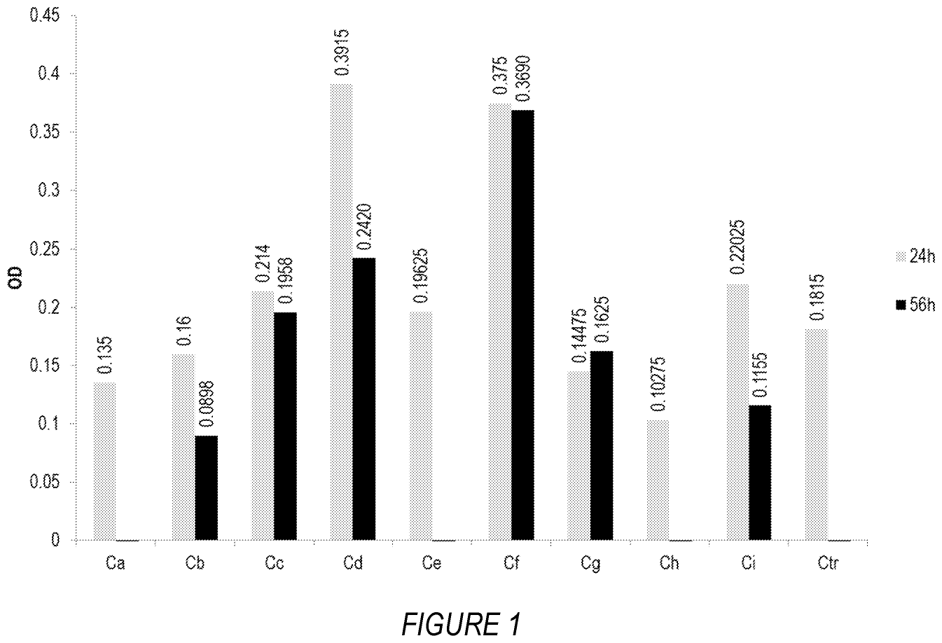

Fish model—Sea bream (Sparus aurata) fish were intraperitoneally (IP) administered (by injection) with 1 μg of the different ODNs or with PBS (for background). Blood was collected 24 hours and 3 days post administration, and total serum IgM level was measured by ELISA analysis in order to evaluate the immunostimulatory effect of the different CpG ODNs (FIG. 1).

The sequences of the different CpG ODNs are provided in Table 1.

TABLE 1

| | | SEQ | | |
|---|---|---|---|---|
| | | ID | | |
| ODNs | Sequence | NO: | # nt | # CG |
| CpG ODN | TCGTCGTTTTCGGCGCGCGCCG | 1 | 22 | 7 |
| Positive | | | | |
| Control Ca | | | | |
| CpG ODN | TCGTCGTN¹TTCGGCGCGCGCCG | 2 | 22 | 7 |
| Cb | | | | |
| CpG ODN Cc | TCGTCGTTN¹TCGGCGCGCGCCG | 3 | 22 | 7 |
| CpG ODN | TCGTCGTCGTTCGAACGACGTTGAT | 4 | 25 | 6 |
| Cd | | | | |
| CpG ODN Ce | AACGTTTCGTTCGAACGACGTTGAT | 5 | 25 | 5 |
| CpG ODN Cf | GACGTTTCGTTCGAACGACGTTGAT | 6 | 25 | 5 |
| CpG ODN | GTCGTCTCGTTCGAACGACGTTGAT | 7 | 25 | 5 |
| Cg | | | | |
| CpG ODN | GTCGTTTCGTTCGAACGACGTTGAT | 8 | 25 | 5 |
| Ch | | | | |
| CpG ODN Ci | AACGTTTCGTTCGAACGTTGTTGAT | 9 | 25 | 4 |
| Non-CpG | TGCTCCTGGAGGGGTTGT | 10 | 18 | 0 |
| ODN | | | | |
| Negative | | | | |
| Control | | | | |

Conclusion: As can be seen in FIG. 1 and in Table 2, all the CpG ODNs tested had a significant short-term effect, with Cc, Cd, Ce, Cf and Ci having a superior effect compared to the negative control Non-CpG ODN (0.1815 at 24 hours). CpG ODNs Cb, Cc, Cd, Cf, Cg and Ci also showed a significant long-lasting effect, having a superior effect compared to the negative control Non-CpG ODN (0 at 56 hours).

TABLE 2

| | | SEQ | | |
|---|---|---|---|---|
| CpG | | ID | 24 | 56 |
| ODNs | Sequence | NO: | hours | hours |
| Ca | TCGTCGTTTTCGGCGCGCGCCG | 1 | + | − |
| Cb | TCGTCGTNTTCGGCGCGCGCCG | 2 | + | + |
| Cc | TCGTCGTTNTCGGCGCGCGCCG | 3 | + | + |
| Cd | TCGTCGTCGTTCGAACGACGTTGAT | 4 | + | + |
| Ce | AACGTTTCGTTCGAACGACGTTGAT | 5 | + | − |
| Cf | GACGTTTCGTTCGAACGACGTTGAT | 6 | + | + |
| Cg | GTCGTCTCGTTCGAACGACGTTGAT | 7 | + | + |
| Ch | GTCGTTTCGTTCGAACGACGTTGAT | 8 | + | − |
| Ci | AACGTTTCGTTCGAACGTTGTTGAT | 9 | + | + |

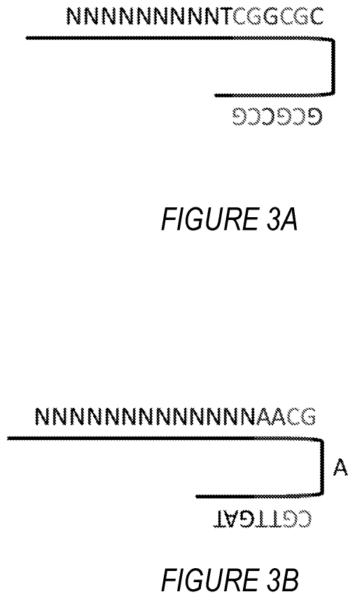

FIG. 3A illustrates a hypothetical internal palindrome sequence and the internal GC dinucleotides in CpG ODNs Ca to Cc. FIG. 3B illustrates a hypothetical palindrome sequence and the internal GC dinucleotides in CpG ODNs Cd to Ci.

### Example 2—CpG ODN Administration Increases Expression of Innate and Adaptive Immune System-Related Genes in Sea Bream

CpG ODNs—The experiment described in Example 1 was repeated with 4 of the CpG ODNs that showed a prolonged elevation (over time) of total IgM in fish serum, and therefore have the best potential to act as long-term immunostimulants.

Fish model—Sea bream (Sparus aurata) fish were administered (by injection) both IP and intradermally (ID) with Cc, Cd, Cf, Cg and PBS control. Serum samples were collected from the IP injected fish as described before, with the exception of adding two more sampling time points, at 7 days and 14 days post administration. In addition, head kidney and spleen samples were collected for Nano string analysis at the same time points. Local skin biopsy samples were collected from the site of administration from fish injected intradermally with the different CpG ODNs.

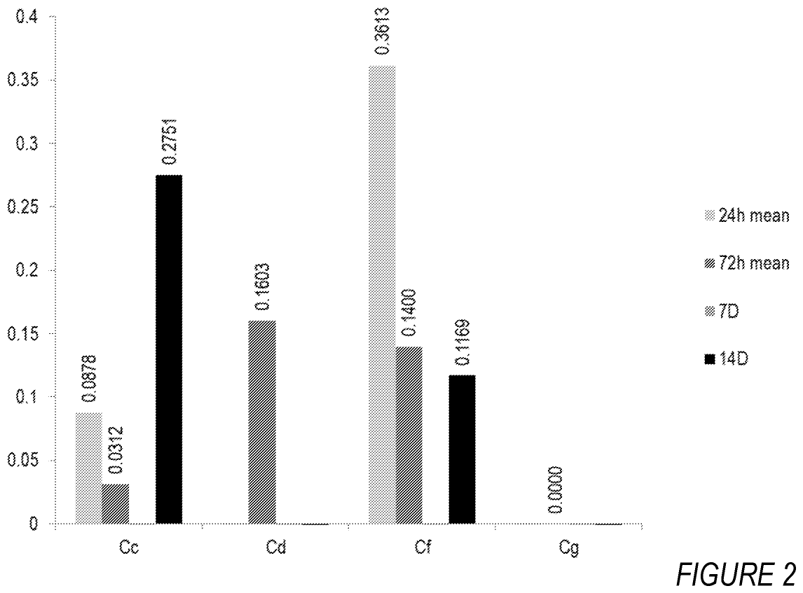

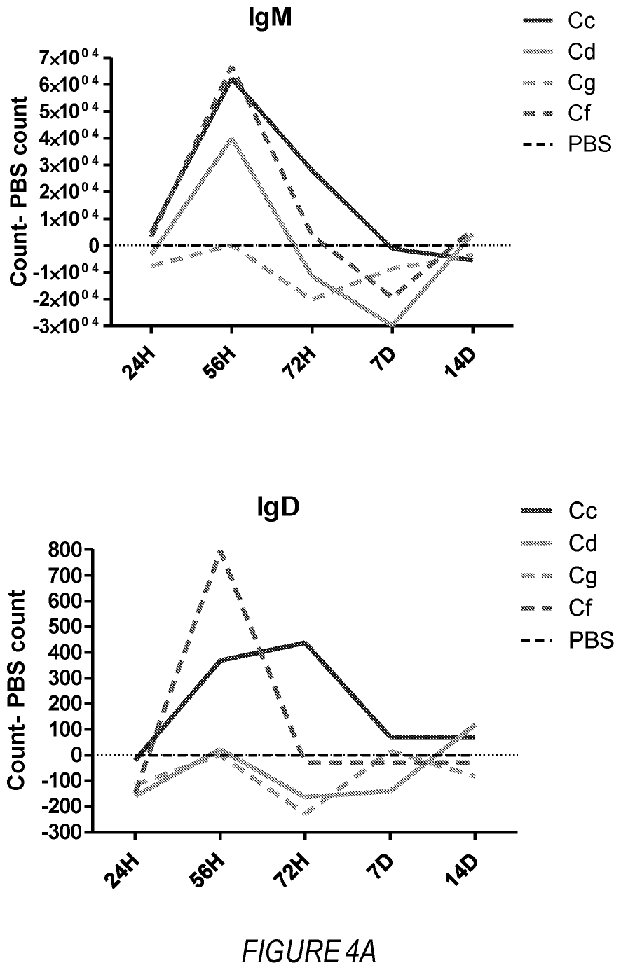

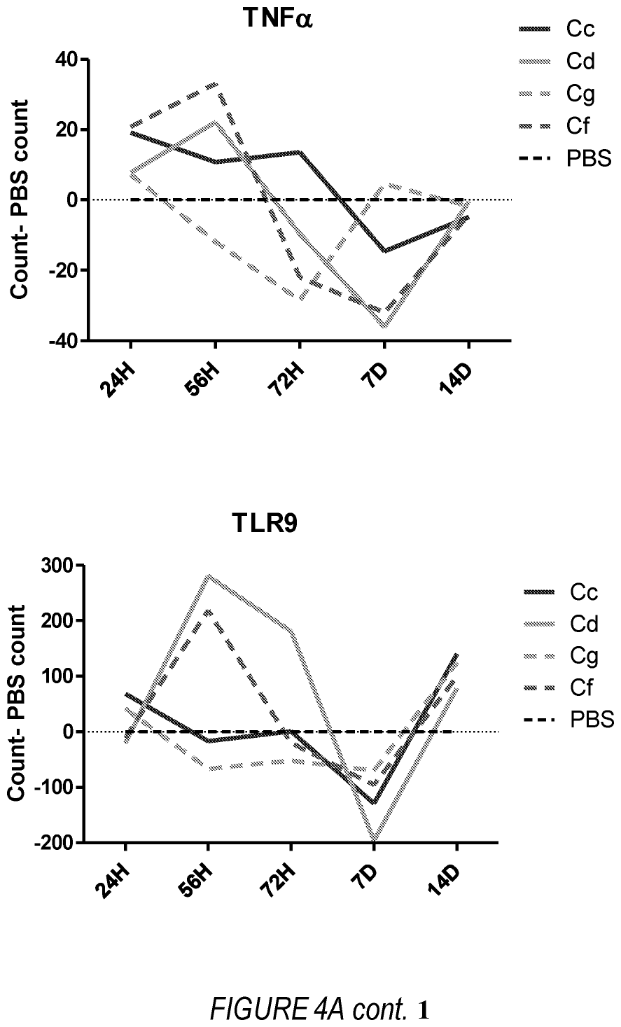

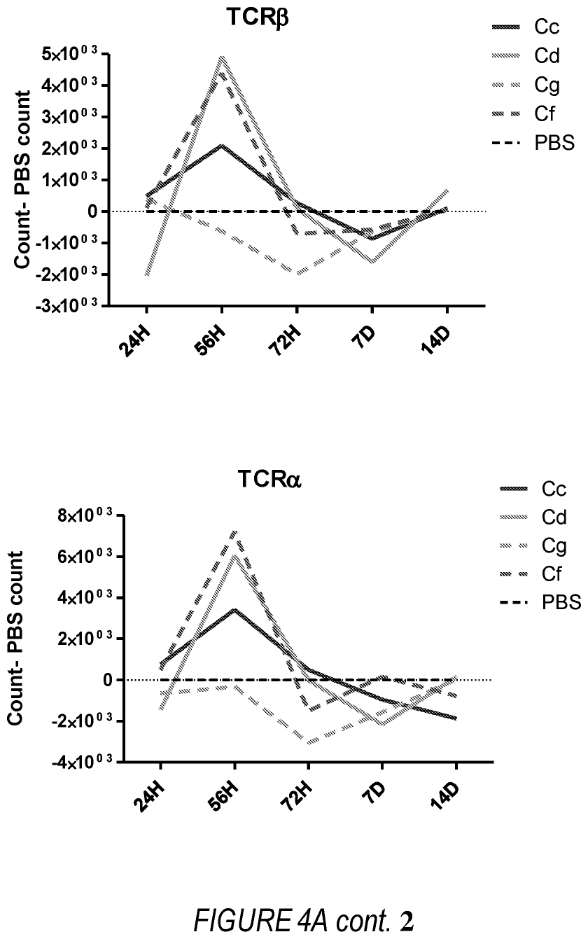

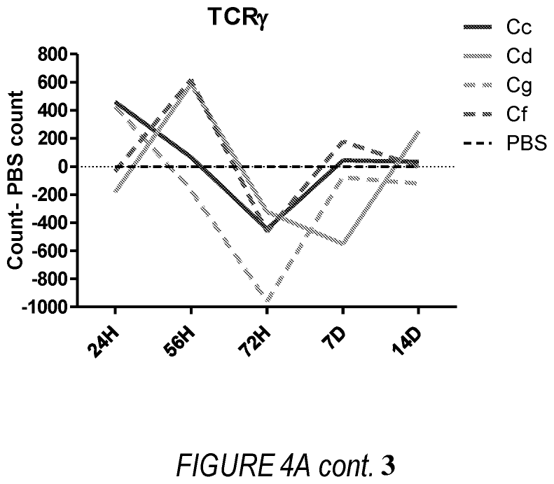

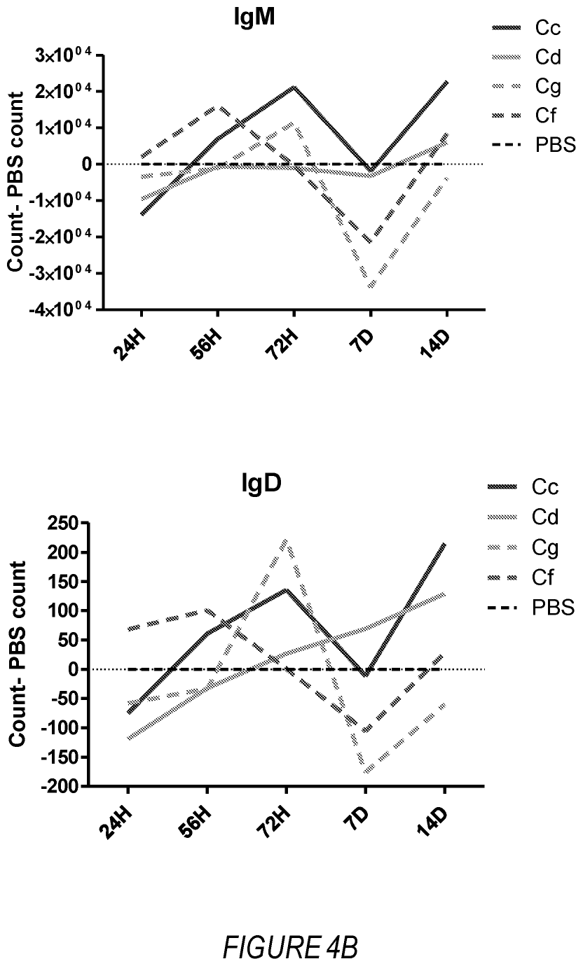

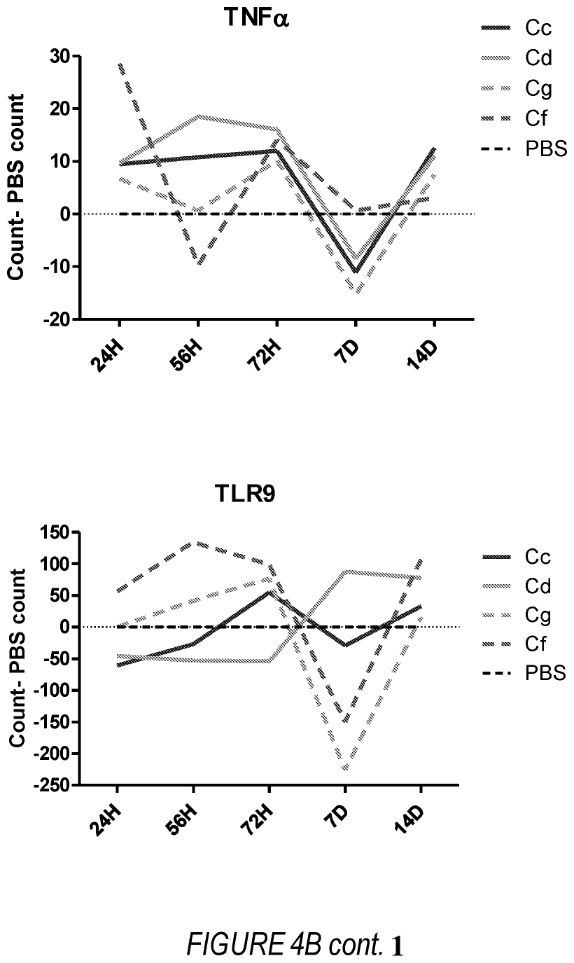

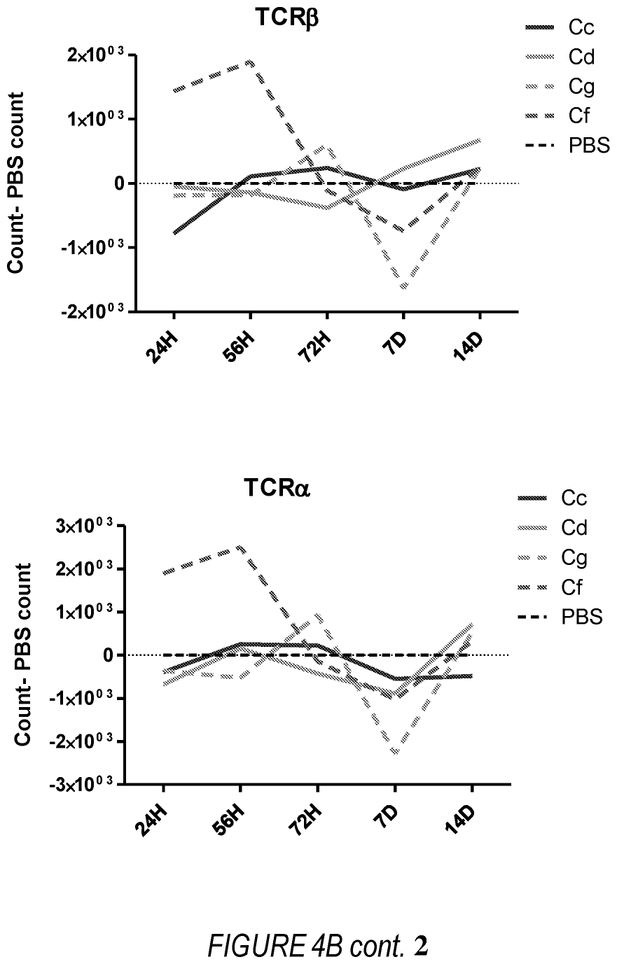

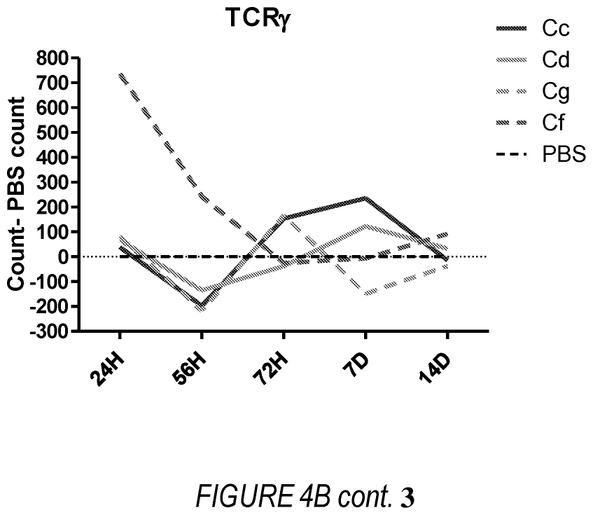

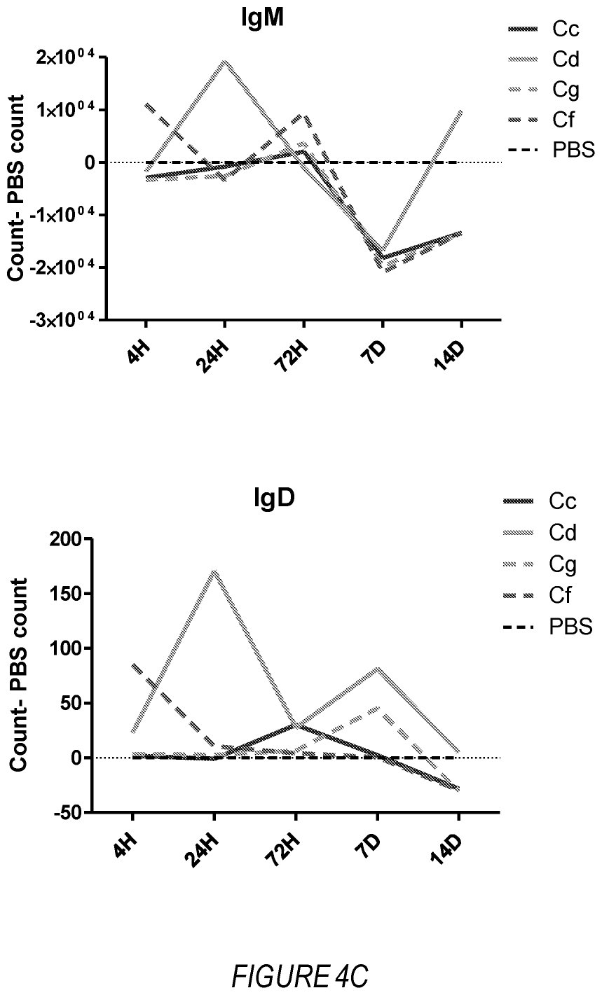

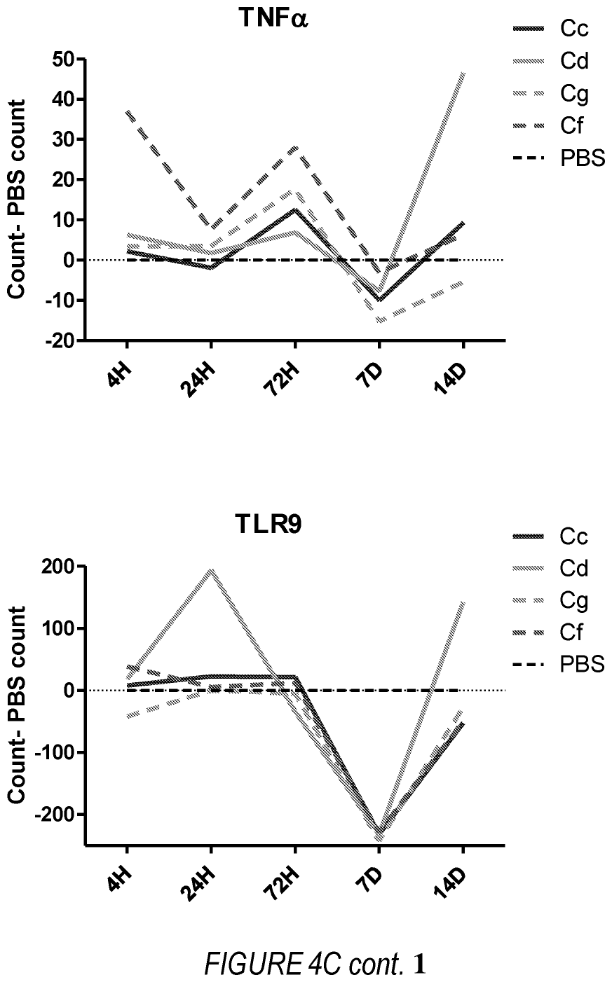

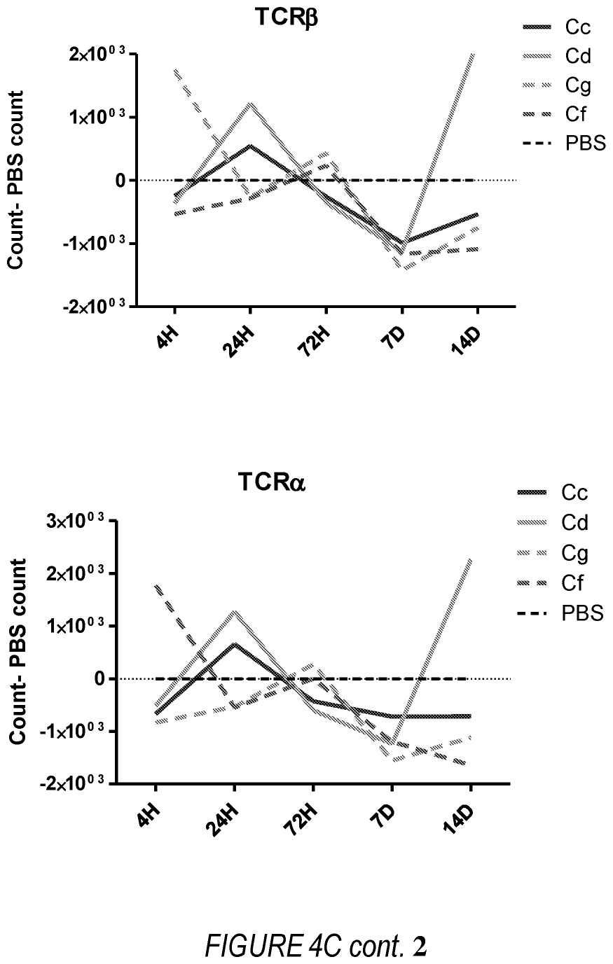

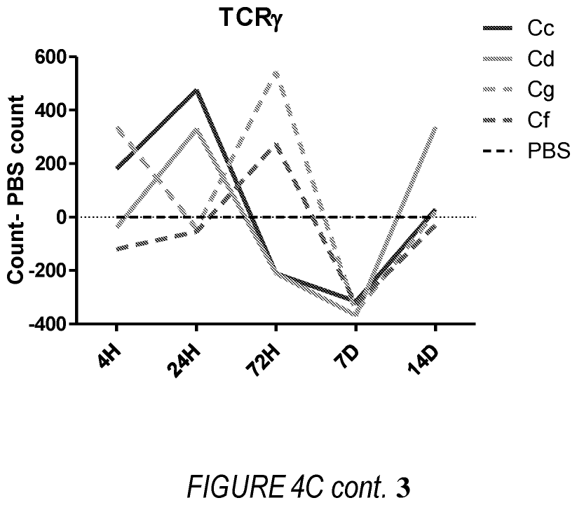

Total IgM in the serum samples was measured by ELISA (FIG. 2), and Nano string analysis evaluated the up/down regulation of specific Sparus aurata immune genes from spleen (Table 3A, FIG. 4A), head kidney (Table 3B, FIG. 4B) and skin (Table 3C, FIG. 4C) biopsy samples. Analysis was normalized to control groups injected with PBS only (OD presented are OD measured minus OD of the control).

Conclusion: The results in FIG. 2 show that fish injected with Cf-CpG have an increase in total antibodies (IgMs) that reach a maximum level after 24 hours and which remained high relative to the control group even 14 days after injection. Fish injected with Cd-CpG, had an increase in total antibodies 72 hours post injection. Examination of the total antibodies in fish injected with Cc-CpG found an increase antibody level as early as 24 hours after injection, which reached a maximum at 14 days after injection in comparison to the control group. Cg-CpG injected fish did not show

TABLE 3A
TLR9 spleen

| | Cc | Cd | Cg | Cf | PBS | Ci |
|---|---|---|---|---|---|---|
| 24H | 179.35 | 91.06 | 152.76 | 100.27 | 111.38 | |
| 72H | 190.13 | 370.15 | 136.9 | 168.85 | 188.97 | |
| 7D | 119.11 | 52.61 | 178.39 | 152.93 | 248.66 | |
| 14D | 263.92 | 201.49 | 247.67 | 222.1 | 123.57 | |
| 56H | 59.74 | 356.87 | 9.89 | 295.1 | 76.58 | 271.01 |

TNFα spleen

| | Cc | Cd | Cg | Cf | PBS | Ci |
|---|---|---|---|---|---|---|
| 24H | 31.62 | 20.2 | 19.65 | 33.19 | 12.45 | |
| 72H | 57.07 | 33.87 | 14.95 | 21.52 | 43.48 | |
| 7D | 38.88 | 17.43 | 57.99 | 21.46 | 53.47 | |
| 14D | 20.64 | 25.03 | 23.69 | 21.28 | 25.48 | |
| 56H | 25.14 | 36.53 | 2.58 | 47.35 | 14.4 | 61.78 |

IgD spleen

| | Cc | Cd | Cg | Cf | PBS | Ci |
|---|---|---|---|---|---|---|
| 24H | 148.55 | 9.58 | 53.25 | 22.01 | 169.84 | |
| 72H | 672.3 | 70.54 | 5.36 | 204.36 | 234.67 | |
| 7D | 221.56 | 10.4 | 164.53 | 121.62 | 150.35 | |
| 14D | 160.99 | 207.08 | 7.93 | 60.73 | 90.87 | |
| 56H | 370.53 | 23.71 | 2.44 | 798.1 | 2.83 | 122.39 |

IgM spleen

| | Cc | Cd | Cg | Cf | PBS | Ci |
|---|---|---|---|---|---|---|
| 24H | 25637.71 | 17361.33 | 13099.74 | 23978.01 | 20692.98 | |
| 72H | 61076.41 | 21674.83 | 12802 | 36957.53 | 33011.2 | |
| 7D | 32789.08 | 4122.88 | 25199.07 | 14532.35 | 33801.48 | |
| 14D | 22454.15 | 32168.43 | 24356.77 | 33885 | 27787.97 | |
| 56H | 64211.49 | 42001.1 | 2080.85 | 68706.16 | 1922.29 | 31616.67 |

TABLE 3B
TLR9 head kidney

| | Cc | Cd | Cg | Cf | PBS | Ci |
|---|---|---|---|---|---|---|
| 24H | 171.57 | 186.65 | 232.4 | 288.23 | 232.22 | |
| 72H | 140.63 | 31.59 | 162.03 | 184.37 | 85.48 | |
| 7D | 214.65 | 331.48 | 17.52 | 93.93 | 243.54 | |
| 14D | 242.44 | 286.69 | 225.29 | 315.82 | 209.3 | |
| 56H | 62.14 | 36.07 | 130.39 | 223.15 | 88.98 | 74.97 |

TNFα head kidney

| | Cc | Cd | Cg | Cf | PBS | Ci |
|---|---|---|---|---|---|---|
| 24H | 15.6 | 15.79 | 12.79 | 34.7 | 6.12 | |
| 72H | 14.78 | 18.83 | 12.97 | 16.86 | 2.81 | |
| 7D | 11 | 13.57 | 6.82 | 22.73 | 22.09 | |
| 14D | 20.25 | 18.74 | 15.29 | 10.81 | 7.73 | |
| 56H | 29.76 | 37.49 | 19.66 | 9.27 | 19 | 45.46 |

IgD head kidney

| | Cc | Cd | Cg | Cf | PBS | Ci |
|---|---|---|---|---|---|---|
| 24H | 78.76 | 34.32 | 95.21 | 221.25 | 153.16 | |
| 72H | 136.41 | 28.11 | 222.35 | 1.55 | 1 | |
| 7D | 166.51 | 246.79 | 1.34 | 72.17 | 177.83 | |
| 14D | 290.45 | 205.12 | 15.71 | 103.35 | 76.02 | |
| 56H | 96.5 | 3.39 | 2.21 | 136.21 | 35.58 | 34.26 |

IgM head kidney

| | Cc | Cd | Cg | Cf | PBS | Ci |
|---|---|---|---|---|---|---|
| 24H | 11020.68 | 15352.35 | 21526.83 | 26908.06 | 24999.23 | |
| 72H | 26957.67 | 4678.37 | 17067.4 | 5233.14 | 5709.39 | |
| 7D | 35245.76 | 33879.26 | 3401.64 | 15699.2 | 37160.61 | |
| 14D | 47578.61 | 30811.22 | 20999.3 | 33194.25 | 24827.08 | |
| 56H | 16559.26 | 8929.14 | 8406.48 | 25607.44 | 9577.84 | 15574.24 |

TCRα head kidney

| | Cc | Cd | Cg | Cf | PBS | Ci |
|---|---|---|---|---|---|---|
| 24H | 1201.03 | 922.91 | 1230.67 | 3488.46 | 1592.99 | |
| 72H | 968.84 | 317.01 | 1669.51 | 598.51 | 739.88 | |
| 7D | 2010.45 | 1668.75 | 268.99 | 1526.7 | 2552.57 | |
| 14D | 1402.67 | 2594.78 | 2401.42 | 2198.77 | 1878.91 | |
| 56H | 1212.4 | 1115.91 | 431.58 | 3455.24 | 954.45 | 1473.35 |

TCR▭ head kidney

| | Cc | Cd | Cg | Cf | PBS | Ci |
|---|---|---|---|---|---|---|
| 24H | 997.29 | 1731.9 | 1590.72 | 3213.88 | 1780.25 | |
| 72H | 913.3 | 293.81 | 1275.73 | 561.29 | 672.96 | |
| 7D | 1789.09 | 2109.83 | 243.57 | 1140.08 | 1880.57 | |
| 14D | 1675.96 | 2127.66 | 1693.61 | 1714.82 | 1452.56 | |
| 56H | 1084.81 | 833.16 | 788.14 | 2869.22 | 974.7 | 1335.96 |

[symbol]

head kidney

| | Cc | Cd | Cg | Cf | PBS | Ci |
|---|---|---|---|---|---|---|
| 24H | 352.97 | 382.21 | 396.16 | 1050.51 | 315.45 | |
| 72H | 268.59 | 75.68 | 279.22 | 88.21 | 114.01 | |
| 7D | 443.76 | 330.12 | 58.99 | 201.71 | 208.43 | |
| 14D | 256.6 | 303.76 | 233.24 | 363.04 | 270.97 | |
| 56H | 120.05 | 182.42 | 101.6 | 559.35 | 317.31 | 183.87 |

TABLE 3C
TLR9 skin

| | Cc | Cd | Cg | Cf | PBS | Ca | Cb | Ce | Ch | Ci | Ctr |
|---|---|---|---|---|---|---|---|---|---|---|---|
| 24H | 44.19 | 215.13 | 21.19 | 26.76 | 21.73 | | | | | | |
| 72H | 83.15 | 27.22 | 56.1 | 73.49 | 61.53 | | | | | | |
| 7D | 31.92 | 32.08 | 21.16 | 34.92 | 263.71 | | | | | | |
| 14D | 19.23 | 213.93 | 43.95 | 21.44 | 71.79 | | | | | | |
| 24H_1 | 217.18 | 194.93 | 93.48 | 38.13 | 96.5 | 28.67 | 232.07 | 92.38 | 89.95 | 89.5 | 90.66 |
| 24H_0.5 | 35.42 | 69.8 | 33.01 | 102.23 | | | | | | | |
| 4H_1 | 57.85 | 68.08 | 7.57 | 88.79 | 50.1 | | | | | | |
| 4H_0.5 | 36.58 | 35.86 | 22.69 | 121.2 | | | | | | | |

TNFα skin

| | Cc | Cd | Cg | Cf | PBS | Ca | Cb | Ce | Ch | Ci | Ctr |
|---|---|---|---|---|---|---|---|---|---|---|---|
| 24H | 9.36 | 13.02 | 14.75 | 18.84 | 11.27 | | | | | | |
| 72H | 28.49 | 22.82 | 33.55 | 43.95 | 15.98 | | | | | | |
| 7D | 7.58 | 9.77 | 2.29 | 14.46 | 17.55 | | | | | | |
| 14D | 19.23 | 56.45 | 4.44 | 16.21 | 9.91 | | | | | | |
| 24H_1 | 29.3 | 8.79 | 16.97 | 3.07 | 10.12 | 4.17 | 1.78 | 5.3 | 11.09 | 4.84 | 16.77 |
| 24H_0.5 | 2.02 | 13.52 | 11.14 | 15.45 | | | | | | | |
| 4H_1 | 6.34 | 10.5 | 7.57 | 41.17 | 4.23 | | | | | | |
| 4H_0.5 | 34.96 | 39.01 | 2.32 | 31.48 | | | | | | | |

IgD skin

| | Cc | Cd | Cg | Cf | PBS | Ca | Cb | Ce | Ch | Ci | Ctr |
|---|---|---|---|---|---|---|---|---|---|---|---|
| 24H | 1.09 | 172.74 | 4.62 | 12.51 | 1.97 | | | | | | |
| 72H | 37.45 | 34.92 | 13.66 | 11.46 | 7.37 | | | | | | |
| 7D | 3.48 | 82.29 | 46.32 | 1.46 | 1 | | | | | | |
| 14D | 4.27 | 37.37 | 1.8 | 2.61 | 32.41 | | | | | | |
| 24H_1 | 2.65 | 100.55 | 102.88 | 3.07 | 2.54 | 2.72 | 1.78 | 4.15 | 3.03 | 2.73 | 1.61 |
| 24H_0.5 | 2.02 | 1.06 | 3.12 | 6.09 | | | | | | | |
| 4H_1 | 6.34 | 27.43 | 7.57 | 89.42 | 4.23 | | | | | | |
| 4H_0.5 | 12.37 | 3.15 | 2.32 | 189.1 | | | | | | | |

IgM skin

| | Cc | Cd | Cg | Cf | PBS | Ca | Cb | Ce | Ch | Ci | Ctr |
|---|---|---|---|---|---|---|---|---|---|---|---|
| 24H | 3151.29 | 23304.59 | 1327.01 | 574.48 | 3949.82 | | | | | | |
| 72H | 6109.93 | 3064.1 | 7764.42 | 13464.78 | 4064.84 | | | | | | |
| 7D | 6412.37 | 7878.45 | 4818.01 | 3604.86 | 24562.92 | | | | | | |
| 14D | 1821.74 | 25050.75 | 1784.42 | 1832.49 | 15214.39 | | | | | | |
| 24H_1 | 18690.57 | 18271.7 | 15814.06 | 1012.66 | 6641.51 | 1498.54 | 6484.29 | 8456.24 | 6711.37 | 3710.6 | 4342.68 |
| 24H_0.5 | 8756.5 | 3620.12 | 2629.56 | 11485.09 | | | | | | | |
| 4H_1 | 1778.06 | 2956.98 | 1376.06 | 15758.73 | 4679.95 | | | | | | |
| 4H_0.5 | 3670.52 | 3461.52 | 2362.76 | 22287.09 | | | | | | | |

TCRα skin

| | Cc | Cd | Cg | Cf | PBS | Ca | Cb | Ce | Ch | Ci | Ctr |
|---|---|---|---|---|---|---|---|---|---|---|---|
| 24H | 1402.39 | 2017.43 | 210.9 | 200.89 | 750.62 | | | | | | |
| 72H | 767.72 | 594.98 | 1465.69 | 1191.4 | 1197.77 | | | | | | |
| 7D | 1294.1 | 776.83 | 455.78 | 818.17 | 2015.38 | | | | | | |
| 14D | 1535.56 | 4514.85 | 1127.03 | 598.99 | 2248.68 | | | | | | |
| 24H_1 | 2574.98 | 1588.64 | 1226.59 | 136.51 | 658.01 | 137.55 | 1443.38 | 1062.7 | 481.23 | 398.08 | 990.22 |
| 24H_0.5 | 1515.56 | 899.24 | 320.47 | 1352.06 | | | | | | | |
| 4H_1 | 678.29 | 833.48 | 513.51 | 3118.27 | 1349.34 | | | | | | |
| 4H_0.5 | 954.75 | 1022.27 | 493.49 | 2405.44 | | | | | | | |

TCR▭ skin

| | Cc | Cd | Cg | Cf | PBS | Ca | Cb | Ce | Ch | Ci | Ctr |
|---|---|---|---|---|---|---|---|---|---|---|---|
| 24H | 1402.39 | 2017.43 | 210.9 | 200.89 | 750.62 | | | | | | |
| 72H | 767.72 | 594.98 | 1465.69 | 1191.4 | 1197.77 | | | | | | |
| 7D | 1294.1 | 776.83 | 455.78 | 818.17 | 2015.38 | | | | | | |
| 14D | 1535.56 | 4514.85 | 1127.03 | 598.99 | 2248.68 | | | | | | |
| 24H_1 | 2574.98 | 1588.64 | 1226.59 | 136.51 | 658.01 | 137.55 | 1443.38 | 1062.7 | 481.23 | 398.08 | 990.22 |
| 24H_0.5 | 1515.56 | 899.24 | 320.47 | 1352.06 | | | | | | | |
| 4H_1 | 678.29 | 833.48 | 513.51 | 3118.27 | 1349.34 | | | | | | |
| 4H_0.5 | 954.75 | 1022.27 | 493.49 | 2405.44 | | | | | | | |

[symbol]

skin

| | Cc | Cd | Cg | Cf | PBS | Ca | Cb | Ce | Ch | Ci | Ctr |
|---|---|---|---|---|---|---|---|---|---|---|---|
| 24H | 562.22 | 415.53 | 43.29 | 31.51 | 86.83 | | | | | | |
| 72H | 207.7 | 207.67 | 963.11 | 692.25 | 418.53 | | | | | | |
| 7D | 132.76 | 79.5 | 92.07 | 119.67 | 447.93 | | | | | | |
| 14D | 249.88 | 558.19 | 239.73 | 191.31 | 220.85 | | | | | | |
| 24H_1 | 177.49 | 370.06 | 38.13 | 235.82 | 35.52 | 25.95 | 141.35 | 55.06 | 35.35 | 51.26 | 164.55 |
| 24H_0.5 | 67.77 | 71.93 | 219.64 | 26.76 | | | | | | | |
| 4H_1 | 315.44 | 95.17 | 471.63 | 14.15 | 134.74 | | | | | | |
| 4H_0.5 | 154.37 | 108.34 | 705.6 | 106.18 | | | | | | | |

Conclusion: the results in FIGS. 4A-C and Tables 3A-C, show that fish injected with Cf-CpG, had an increase in spleen and head kidney gene expression, which regulate IgD and IgM for up to 56 hours after injection. IgD and IgM gene expression increased to a maximum 72 hours after injection. In addition, these fish also showed an increase in all genes tested belonging to the innate immune system (TNFα, TLR9, TCRb, TCRb, TCRg) in spleen and kidney. Fish injected with CpG-Cd, had an increase in expression of the IgD gene, which peaked at 72 hours post injection in the spleen, while in the head kidney it remained high even 14 days after injection. The IgM gene expression levels reached a peak increase in the spleen, 56 hours after injection. In addition, these fish also showed an increase in all genes tested belonging to the innate immune system (TNFα, TLR9, TCRb, TCRb, TCRg) in spleen and kidney 72 hours post injection. Fish injected with Cc-CpG, showed an increase in the expression of IgM gene in spleen, reaching a peak after 56 hours, while in the head kidney the peak was 72 hours after injection. The expression of genes for the formation of IgD, was seen to increase only in the head kidney at 72 hours post injection. Fish injected with Cg-CpG, showed an increase in the expression of genes for IgM and IgD in the kidney, reaching a maximum level 72 hours after injection.
Subcutaneous injection (skin) also appeared to increase gene expression in all four CpGs at different times. To maximize immediate and maintain a long-term efficacy of the CpGs on fish immunity, the combination of a few or all CpG's should be considered.

### Example 3—CpG ODN Administration Increases Total IgM Levels in Fish

Fish are administered five different doses of four types of CpG ODNs:

Cc-
(SEQ ID NO: 3)
TCGTCGTTNTCGGCGCGCGCCG;
Cd-
(SEQ ID NO: 4)
TCGTCGTCGTTCGAACGACGTTGAT;
Cf-
(SEQ ID NO: 6)
GACGTTTCGTTCGAACGACGTTGAT;
and
Cg-
(SEQ ID NO: 7)
GTCGTCTCGTTCGAACGACGTTGAT.

The immune response offish is followed throughout the experiment, compared with a control group injected with PBS solution. Blood is collected from each fish at the beginning of the experiment, after 72 hours and at 7, 14, 30, 60 days post administration, and the level of antibodies is monitored. Blood samples are refrigerated, left to clot at 4° C. overnight, centrifuged (3500 g, 30 min) and the serum is subsequently stored at −20° C. until further analysis.

To quantify total Sparus aurata IgM titer, non-competitive ELISA serological analyses is performed. A 96-well ELISA plate is coated with 100 μl/well of fish serum (diluted 2×10⁻³ in PBS), incubated for 1 h at room temperature (RT) and later incubated at 4° C. overnight (ON). The next day, fluids are discarded, and the plate is rinsed once with 250 μl/well of washing buffer (PBS-T). The plate is then coated with 250 μl/well of blocking solution (1% gelatin in PBS), incubated for 2 hours at RT, and a single brief wash follows. Then, 100 μl of mouse anti-Sparus aurata (Aquatic Diagnostics, Ltd., Sterling, U.K.) suspended the day before in 1 ml PBS, and diluted again 5×10⁻³ (Normal Goat Serum Gibco®, Applied Biosystems) in PBS is added to all the wells and incubated for 1 hour at RT. The fluids are discarded, and the plate is rinsed (3×5 min). 100 μl of Goat Anti-mouse IgG-HRP conjugate (Bio-Rad Laboratories, Hercules, CA, USA), diluted 10⁻³ in 1% NGS, is added to the entire plate and incubated for 1 hour at RT. Fluids are discarded and the plate is rinsed (3×5 min). 100 μl of SureBlue™ TMB Microwell Peroxidase substrate (Thermo Scientific) is added to all the wells and the plate, wrapped in aluminum foil, is allowed to react for 15 min at RT. Finally, 50 μl/well of stop solution (2M H₂SO₄) is added. Plates are read at 450 nm absorbance in microplate spectrophotometer (Bio-Tek® reader GeneQuant-Pro, Amersham, Cambridge, U.K.). Antibody titers in fish serum are quantified and expressed as OD measurements, minus average OD measurement of PBS injected fish.

125 fish were tagged with a P-tag and distributed equally into five containers (25 fish per container). Each fish (five fish per CpG) from the same container was injected by IP with one of the four different CpGs with the same dose, with a total of five different doses: 2.5 μg, 5 μg, 10 μg, 15 μg, 20 μg (five containers overall). Five fish in each container were injected with PBS and were not tagged. Blood/serum for antibody (IgM) testing was collected from each fish before experiment started (T0), after 72 hours (3 days), and at four additional time points 7, 14, 30, 60 days after injection (3/7/14/30/60D).

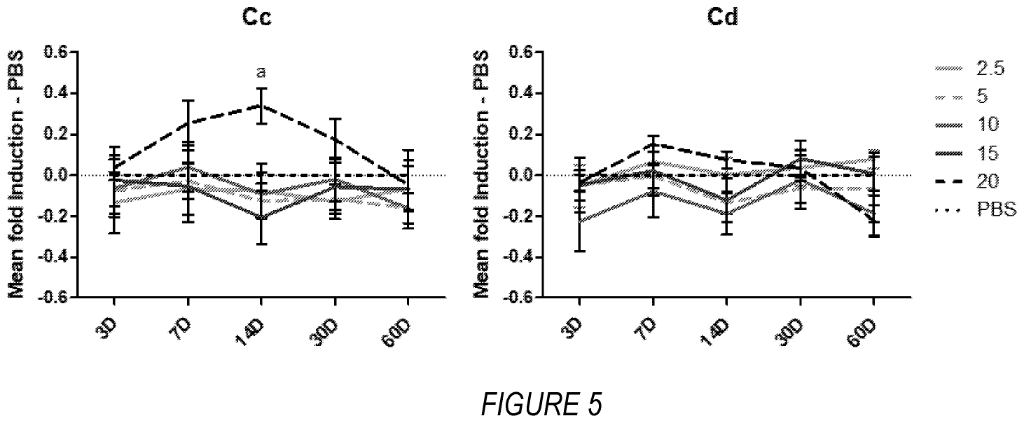

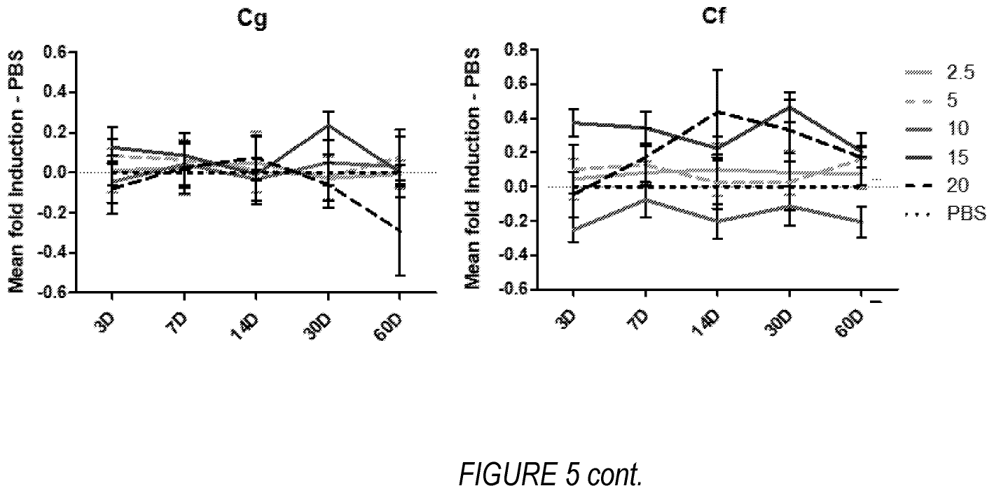

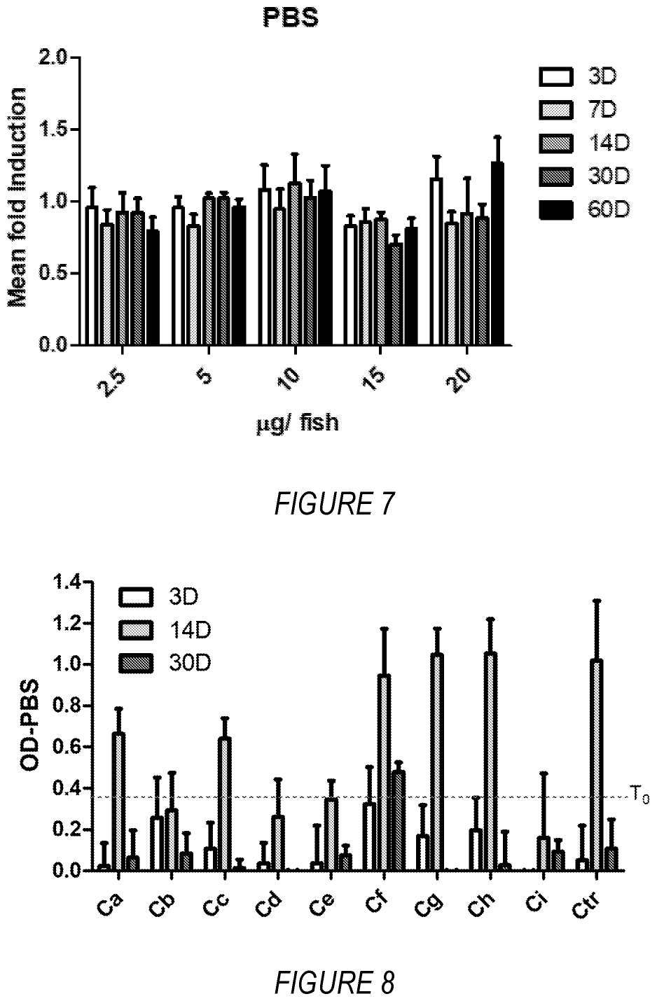

Experimental results are shown in FIGS. 5-7. The amount of antibody obtained following the injection was measured using the ELISA test. Blood samples from each fish was normalized to a fixed sample which was added to each plates (fold induction). After normalization, the mean value of the amount of antibody of fish injected with PBS was decreased (fold induction-PBS). In order to diminish the tank effect, every 5 fish in a treatment tank were injected with PBS as a control group. Accordingly, for each fish result was obtained by subtraction of the average result from the control group (PBS injected fish) from the same tank. It can be seen that no significant differences were observed in the antibody (IgM) levels in fish injected with PBS (control) between the different tanks over time (FIG. 7).

A significant difference between the curves overtime in repeated measurements was found in fish injected with Cf-CpG, where injection of 15 μg and 20 μg significantly increased antibody level relative to zero time (FIG. 5).

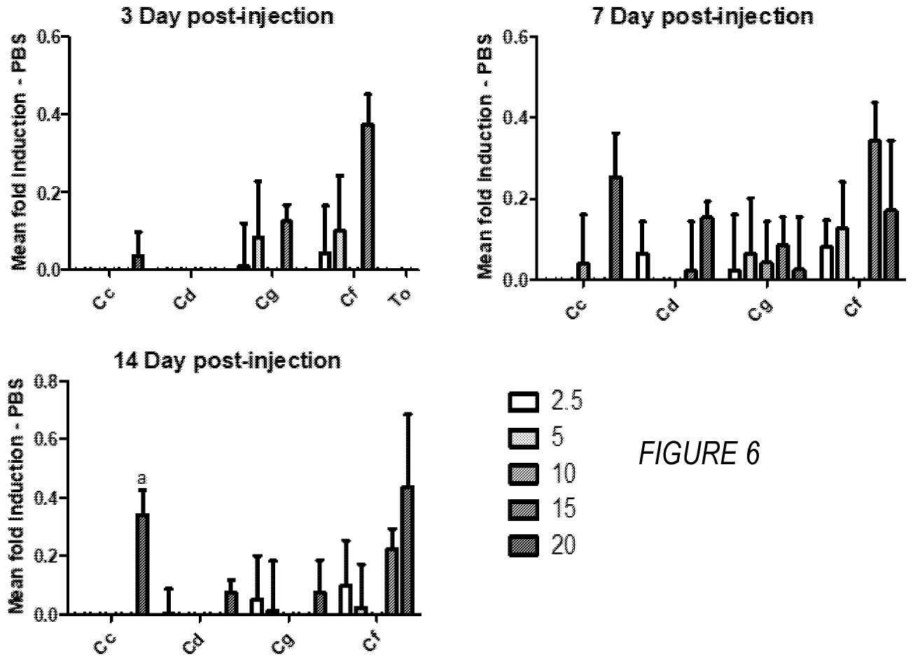

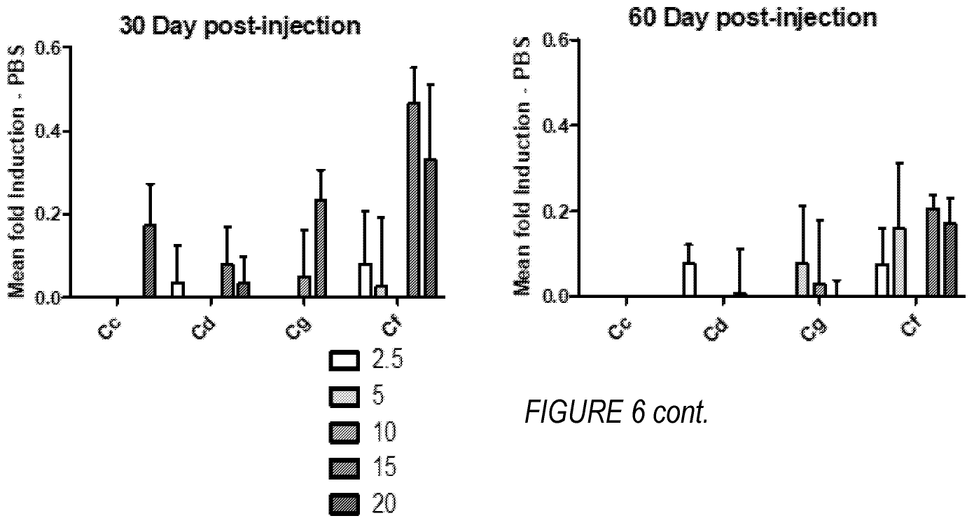

The One Way ANOVA test found that after 14 days there was a significant difference in fish injected with Cc-CpG at a dose of 20 μg over the other doses injected at the same time point (FIG. 6).

### Example 4—CpG ODN Administration Increases Expression of Both Innate and Adaptive Immune System-Related Genes in Fish

Sparus aurata fish are injected intraperitoneally (IP) with four types of CpGs at five different doses compared with control group (PBS). Few fish are sacrificed at the beginning of the experiment, and then from each treatment group at different time points (72 hours and at 14 and 30 days post-injection). Blood, serum, and tissue (head kidney and spleen) are sampled for antibody analysis (ELISA) and for TNFα, TLR9, TCR, IgM, IgD gene quantification. A qPCR assay is performed to quantify five immune genes (TNFα, TLR9, TCR, IgM, IgD) that represent the activation of both parts of the immune system (innate and adaptive).

qPCR assay: Total RNA was extracted from spleen and head-kidney (50-100 mg) target tissues using Bio-Tri RNA Reagent (Bio Lab Ltd., Jerusalem, Israel) according to the manufacturer's instructions and ground by FastPrep-24™ 5G (MP Biomedicals LLC). RNA quantity and purity (260/280 ratio) were estimated in a NanoDrop™ One Microvolume UV-Vis Spectrophotometer (Thermo Fisher Scientific, Madison, WI). DNA residues from the extracted RNA were removed by using DNase 1 (RNase-Free)® kit (Ambion) according to the manufacturer's instructions. Gene expression in spleen and head-kidney tissues was examined using quantitative real-time PCR (RT-qPCR). cDNA from the RNA samples was synthesized by using qScript™ cDNA Synthesis Kit (Quanta BioSciences, Inc., Gaithersburg, MD) according to the manufacturer's protocol. The reaction mix (final volume of 20 l) consisted of 4 μl qScript Reaction Mix (5×), 1 μl qScript RT, 10 μl Nuclease-free water, 4 μg RNA template. The genes transcription level was determined by RT-qPCR, using PerfeCta® qPCR ToughMix™, Low ROX™ (Quanta BioSciences), in 10 μl reaction volume with duplicates. Mix preparation and cycling parameters were set according to the manufacturer's protocol. To rule out possible contaminations, in every reaction a negative control sample containing nuclease-free water was loaded. A constant mixed sample which had shown medium transcription level was also added as positive control. Relative quantification levels were calculated by RQ=2−ΔΔct, CtThreshold cycle. In order to verify efficiency (90-110%) in the expression of both endogenous and target genes, a standard curve was applied for each target gene (IgM, IgD, TNFα, TCRα, TLR9 and βactine) in 6 different serial dilutions, with 3 technical replicates for each sample. Transcription levels were normalized according to the endogenous reference gene βactine. The results were analyzed by 7500 Fast Real-Time PCR system software (Applied Biosystems version 2.0.6).

### Example 5—CpG ODN Administration Increases Total IgM Levels in Sea Bass Serum

The effect of different CpG ODNs on the immune response (IgMs in fish serum) is examined in Dicentrarchus labrax. Different fish species are grouped according to similarity in their immune response to the administered CpG ODNs.

Fish model—Sea bass (Dicentrarchus labrax) fish were intraperitoneally (IP) administered (by injection) with 20 μg of the different ODNs or with PBS (for background). Blood was collected 3, 14, and 30 days post administration, and total serum IgM level was measured by ELISA analysis in order to evaluate the immunostimulatory effect of the different CpG ODNs (FIG. 8). Analysis was normalized to control groups injected with PBS only (OD presented are OD measured minus OD of the control). Nine fish were used in each treatment and experiment was repeated three times (n=27).

Conclusion: The level of total antibodies increased in fish injected with Ca, Cc, Cf, Cg, Ch and control (type C) CpGs and reached a maximum level 14 days after injection. In Cf-CpG injected fish antibody levels remained high even 30 days post injection relative to the PBS injected group.

### Example 6—CpG ODN Administration Increases Total IgM Levels in Sea Bass Serum

Sparus aurata fish are infected with Vibrio harveyi, a gram-negative marine bacterium, following vaccination with composition comprising 4 CpG ODNs. The formation of specific antibodies against the bacterium in the fish was examined following vaccination with 4 different CpG ODNs together with the heat killed bacterium as the antigen.

Fish are administered four types of CpG ODNs:

Cc-
(SEQ ID NO: 3)
TCGTCGTTNTCGGCGCGCGCCG;
Cd-
(SEQ ID NO: 4)
TCGTCGTCGTTCGAACGACGTTGAT;
Cf-
(SEQ ID NO: 6)
GACGTTTCGTTCGAACGACGTTGAT;
and
Cg-
(SEQ ID NO: 7)
GTCGTCTCGTTCGAACGACGTTGAT.

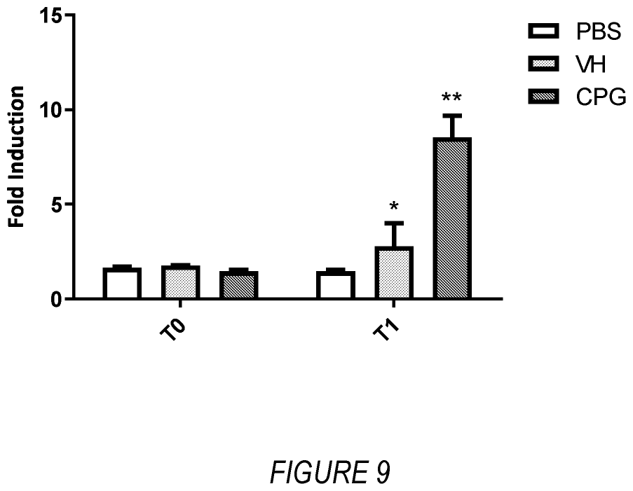

Experimental design: Eighteen fish were equally divided into 3 tanks (6 fish per tank). All fish were labeled by a P-tag. Vaccine group 1 (CpG) fish were injected IP with 100 μl of a CpG composition which consisted of 20 μg from each of the 4 CpGs tested (Cc, Cd, Cf & Cg), 50 μl of oil adjuvant (MONTANIDE™ ISA 263 VG—Seppic S.A.) and 50 μl of heat-killed bacteria (Vibrio harveyi) as the antigen, at a concentration of 4×10⁹ (final injection volume of 200 μl). Vaccine group 2 (VH) fish were injected with 50 μl of oil adjuvant (MONTANIDE™ ISA 263 VG—Seppic S.A.), 50 μl of heat-killed bacteria (Vibrio harveyi) at a concentration of 4×10⁹ and 100 μl of PBS (final injection volume of 200 μl). Control group (PBS) fish were injected with 50 μl of adjuvant (MONTANIDE™ ISA 263 VG—Seppic S.A.) and 150 μl of PBS (final injection volume per fish 200 μl). Three weeks after the initial vaccination, a booster was injected in the same manner as described above. Three weeks after the booster, the fish were IP challenged with 100 μl of live pathogenic bacteria (Vibrio harveyi) at a concentration of 1×10⁹ per fish. Blood and serum were collected from each fish for specific anti Vibrio harveyi antibody testing, at the beginning of the experiment (T0), and before challenge (T1) (FIG. 9). Due to low water temperature measurements (22° c.) no fish mortality was obtained following the injection. Antibody was assayed by multilayer ELISA using Mouse anti-Sea bream IgM monoclonal antibodies (Aquatic Diagnostics, Ltd., Stirling, U.K.), Goat Anti-mouse IgG-HRP conjugate (BioRad Laboratories, Hercules, CA) with a SureBlue™ TMB Microwell Peroxidase substrate (Thermo Fisher Scientific).

Conclusion: As shown in FIG. 9, the fold increase in specific antibodies against Vibrio harveyi was highest in Vaccine group 1 (CpG) fish, which were vaccinated with the composition comprising Cc, Cd, Cf, and Cg and injected together with heat-killed bacteria and to a lesser extent in Vaccine group 2 (VH) fish which were injected with heat-killed bacteria mixed with oil adjuvant, both as compared with control fish (PBS).

The CpG oligodeoxynucleotides (ODN) described herein have been shown to strengthen the self-defense mechanism of fish, thereby providing a method for better controlling and regulating the spread of diseases in fish, for protecting different fish species against a wide range of pathogens and increasing the overall health of fish. The CpG ODNs may also be used as a specific immune enhancer (adjuvant), when combined with a specific antigen (as shown in FIG. 9). Advantageously, due to their small size, the CpG molecules can be concentrated and micro-encapsulated.

1. An immunogenic molecule, comprising an oligonucleotide sequence selected from the group consisting of the oligonucleotide sequence set forth in any one of SEQ ID NO: 2 to SEQ ID NO: 7 and SEQ ID NO: 9, and an immunogenic fragment thereof.

2. The immunogenic molecule of claim 1, wherein the oligonucleotide sequence is unmethylated.

3. The immunogenic molecule of claim 1, wherein the immunogenic molecule further comprises an additional and unmethylated, partly methylated, or fully methylated oligonucleotide sequence.

4-5. (canceled)

6. The immunogenic molecule of claim 1, comprising at least one phosphodiester link, phosphorothioate link, or both between two consecutive CG nucleotides.

7-11. (canceled)

12. The immunogenic molecule of claim 6, wherein the phosphorothioate link includes an organic or inorganic phosphorothioate molecule.

13. (canceled)

14. The immunogenic molecule of claim 12, wherein the inorganic phosphorothioate molecule is a mono-thiophosphate molecule, a di-thiophosphate molecule, a tri-thiophosphate molecule, a tetra-thiophosphate molecule, or a binary thiophosphate/polyphosphate molecule.

15. The immunogenic molecule of claim 1, comprising 6 to 40 nucleotides, 20 to 30 nucleotides, or 22 to 25 nucleotides.

16-17. (canceled)

18. A composition comprising at least one immunogenic molecule, the immunogenic molecule comprising the immunogenic molecule of claim 1.

19. (canceled)

20. The composition of claim 18, wherein the composition is a fish growing composition used to maintain or grow live fish, or the composition is a fish feeding composition used to feed live fish.

21. (canceled)

22. The composition of claim 18, comprising at least about 2.5 μg of the oligonucleotide sequence.

23. (canceled)

24. A method of initiating or promoting innate or adaptive immunity in fish, comprising the step of administering at least one immunogenic molecule to the fish, the immunogenic molecule comprising the immunogenic molecule of claim 1.

25. The method of claim 24, wherein administering the immunogenic molecule to the fish comprises feeding the immunogenic molecule to the fish, adding the immunogenic molecule to the medium of the fish, or contacting the immunogenic molecule with the fish.

26-30. (canceled)

31. The method of claim 24, wherein the administration is repeated at least once every 12 to 36 hours, once every 24 hours, once every 44 to 68 hours, or once every 56 hours.

32-36. (canceled)

37. A method of initiating, promoting or increasing an immune response against an antigen in fish, comprising the step of administering to the fish: (a) at least one immunogenic molecule, comprising the immunogenic molecule of claim 1, and (b) an antigen.

38. (canceled)

39. The method of claim 37, wherein the antigen is a heat-killed bacterium, wherein the bacterium is a gram-negative bacterium.

40. (canceled)

41. The method of claim 37, wherein the bacterium is from:
(a) the Genus selected from the group consisting of Vibrio, Pseudomonas, and Flavobacterium;
(b) the Family selected from the group consisting of Enterobacteriaceae, Vibrionaceae, Pseudomonadaceae, and Flavobacteriaceae: or
the group consisting of Vibrio harvevi, Vibrio parahaemolyticus, Vibrio alginolyticus, Vibrio anguillarum, and Vibrio owensii.

42-44. (canceled)

45. The method of claim 24 or 37, wherein the fish is:
(a) from the genus selected from the group consisting of Sparus, Abramis, Acanthopagrus, Argyrops, Blicca, Brama, Chilotilapia, Dicentrarchus, Oncorhynchus, Etelis, Lepomis, Gymnocranius, Lethrinus, Nemipterus, Pharyngochromis, Rhabdosargus, and Scolopsis;
(b) from the class Actinopterygii;
(c) from the order Perciformes
(d) Seabass (Dicentrarchus labrax); or
(e) Sea bream (Sparus aurata).

46-49. (canceled)

50. The method of claim 24 or 37, comprising administering at least about 2.5 μg of the oligonucleotide sequence to each fish.

51. (canceled)


SEQUENCE LISTING (10 sequences)

SEQ ID NO: 1 - DNA, 22 bp, Artificial Sequence
  Description of Artificial Sequence Synthetic oligonucleotide
tcgtcgttttcggcgcgcgccg

SEQ ID NO: 2 - DNA, 22 bp, Artificial Sequence
  Description of Artificial Sequence Synthetic oligonucleotide
  modified_base (8)..(8): a, t, c, or g
tcgtcgtnttcggcgcgcgccg

SEQ ID NO: 3 - DNA, 22 bp, Artificial Sequence
  Description of Artificial Sequence Synthetic oligonucleotide
  modified_base (9)..(9): a, t, c, or g
tcgtcgttntcggcgcgcgccg

SEQ ID NO: 4 - DNA, 25 bp, Artificial Sequence
  Description of Artificial Sequence Synthetic oligonucleotide
tcgtcgtcgttcgaacgacgttgat

SEQ ID NO: 5 - DNA, 25 bp, Artificial Sequence
  Description of Artificial Sequence Synthetic oligonucleotide
aacgtttcgttcgaacgacgttgat

SEQ ID NO: 6 - DNA, 25 bp, Artificial Sequence
  Description of Artificial Sequence Synthetic oligonucleotide
gacgtttcgttcgaacgacgttgat

SEQ ID NO: 7 - DNA, 25 bp, Artificial Sequence
  Description of Artificial Sequence Synthetic oligonucleotide
gtcgtctcgttcgaacgacgttgat

SEQ ID NO: 8 - DNA, 25 bp, Artificial Sequence
  Description of Artificial Sequence Synthetic oligonucleotide
gtcgtttcgttcgaacgacgttgat

SEQ ID NO: 9 - DNA, 25 bp, Artificial Sequence
  Description of Artificial Sequence Synthetic oligonucleotide
aacgtttcgttcgaacgttgttgat

SEQ ID NO: 10 - DNA, 18 bp, Artificial Sequence
  Description of Artificial Sequence Synthetic oligonucleotide
tgctcctggaggggttgt